(19) United States
(12) Patent Application Publication
(10) Pub. No.: US 2023/0268024 A1
(43) Pub. Date: Aug. 24, 2023

(54) COMPUTATIONAL MODELS TO ANALYZE RNA VELOCITY

(71) Applicant: THE UNIVERSITY OF CHICAGO, Chicago, IL (US)

(72) Inventors: Samantha J. RIESENFELD, Chicago, IL (US); Cheng Frank GAO, Chicago, IL (US); Suriyanarayanan Vaikuntanathan, Chicago, IL (US)

(73) Assignee: THE UNIVERSITY OF CHICAGO, Chicago, IL (US)

(21) Appl. No.: 18/155,625

(22) Filed: Jan. 17, 2023

Related U.S. Application Data
(60) Provisional application No. 63/299,752, filed on Jan. 14, 2022.

(51) Int. Cl. G16B 5/00; G16B 30/10; G16B 25/10

(52) U.S. Cl. CPC G16B 5/00; G16B 30/10; G16B 25/10

(57) ABSTRACT

Aspects of the disclosure relate to the finding that certain modeling of single-cell RNA-sequencing (scRNA-seq) data can provide cell profiles of the analyzed cell population. Certain aspects use algorithms including topic modeling, burst modeling, and data integration to determine the profiles. To apply RNA velocity to more general systems, including immune response studies, aspects herein concern a new approach that can infer the cells and genes associated with distinct active processes via probabilistic topic modeling and uses the results to estimate process-specific velocity parameters.

### CROSS-REFERENCE TO RELATED APPLICATIONS

This application claims the benefit of priority to U.S. Provisional Patent Application Ser. No. 63/299,752, filed Jan. 14, 2022, hereby incorporated by reference in its entirety.

### BACKGROUND OF THE INVENTION

### I. Field of the Invention

This invention relates to the field of biophysics, cellular biology, and precision medicine.

### II. Background

One of the key challenges in single-cell data science, trajectory inference (TI) leverages genome-wide transcriptional profiles to estimate the position of each cell in an underlying, ordered biological process [1-3]. The destructive nature of single-cell RNA-sequencing (scRNA-seq) technologies limits the input data to static snapshots, rather than temporal records. Recent computational innovations glean true dynamic information by exploiting inadvertently captured reads from unspliced pre-mRNA, as well as targeted reads from mature, spliced mRNA, to model the transcriptional kinetics of genes and thereby estimate a time derivative of the transcriptional state, known as RNA velocity [4, 5].

Unlike similarity-based “pseudotime” TI methods (e.g., [6-8], reviewed in [3]), RNA velocity reveals the directions and patterns of complex flows, even within a single time point, and thus also precursor and terminal cell populations. Velocity-based approaches have recently expanded to include features, such as handling multimodal data [9, 10], quantifying uncertainty in dynamics [11, 12], accounting for gene length and transcriptional bursting [13], projecting inferred transitions to lower-dimensions [14], using radial basis functions [15] and latent ordinary differential equations [16] to model transcriptional dynamics, estimating time-varying cell-specific parameters [17], extending TI by removing the need to specify root and/or terminal states [11, 18, 19], and leveraging metabolic labeling to construct and interpret single-cell transcriptomic vector fields [20].

The unique capabilities and possible extensions of RNA velocity make it a potentially powerful tool in the analysis of diverse dynamic biological systems, particularly when there is limited prior knowledge. Yet, despite the advances, the effective application of RNA velocity for TI has been impeded by a lack of robustness and accuracy driven by multiple factors, including heterogeneity in true kinetic rates [21-23], confounding of dynamic processes [5, 22], and limitations of the data processing, transcriptional modeling, and visualizations [24, 25]. The gap between the promise and reality of RNA velocity has largely restricted testing and applications to relatively well-structured developmental systems and cell cycle [3-5, 17, 21, 22, 25, 26].

### SUMMARY OF THE INVENTION

Aspects of the disclosure relate to the finding that certain modeling of single-cell RNA-sequencing (scRNA-seq) data can provide cell profiles of the analyzed cell population. Certain aspects use algorithms including topic modeling, burst modeling, and data integration to determine the profiles.

Certain aspects concern methods of building a model of cellular trajectory in a plurality of cells. In some aspects, the method comprises receiving single cell RNA-sequencing (scRNA-seq) data from each cell in the plurality of cells. The scRNA-seq data may be from any source. In some aspects, the source is a biological sample. In some aspects, the source comprises both spliced and unspliced transcripts. In some aspects, unspliced transcripts have not been depleted from the source. In certain aspects, unspliced transcripts have not been substantially depleted from the source. In certain aspects, the scRNA-seq data comprises transcript reads from both spliced and unspliced transcripts. The source may comprise any population of cells, such as a population of cells from a subject, such as a subject having or suspected of having a disease. The disease may be any disease, such as an immune disease, an infection, a cancer, or a genetic disease. The population of cells may be any population of cells, such as a population of immune cells, progenitor cells, and/or neoplastic cells. In some aspects, the method comprises computing the model for the plurality of cells based on the scRNA-seq data using topic modeling. In some aspects, the method comprises calculating at least one RNA velocity parameter.

In some aspects, the scRNA-seq data is filtered. The scRNA-seq data may be filtered by removing genes that do not have spliced and/or unspliced transcripts in at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more (or any range derivable therein), cells of the plurality of cells. The spliced and/or unspliced transcripts can be determined by whether the transcript read has or does not have intronic sequences.

In some aspects, the topic modeling comprises applying a topic model to the scRNA-seq data. The topic model may be any model useful for determining topics, including a multinomial topic model. In some aspects, wherein the topic modeling comprises drawing xi for each cell in the plurality of cells using Equation 1:

xᵢ₁, . . . ,xᵢ₁|tᵢ˜Multinom(tᵢ;π_{i,1}, . . . π_{i,M})∀1≤i≤C (1)

where C is the number of cells, M is the number of genes, xim is number of mRNA transcripts for the mth gene in the ith cell and tᵢ=Σₘ₌₁^{M}xᵢₘ is total number of transcripts in cell i. In certain aspects, multinominal probabilities are calculated using Equation 2:

π_{i,M}=Σₖ₌₁^{K}LᵢₖFₘₖ (2)

where K is the number of user-specified topics (or gene programs), L∈R^{C×K} is the cell topic weight matrix, Lᵢₖ is the proportion that topic k contributes to the counts in cell i; F∈R^{m×K} is the gene program matrix and Fₘₖ is the probability of gene m occurring in topic k. In some aspects, the optimal number of topics, which may be analogous to cellular processes, is calculated. The optimal number may be calculated using statistical modeling. In some aspects, the number of topics is 1, 2, 3, 4, 5, 6, 7, 8, 9, 10, 11, 12, 13, 14, 15, 16, 17, 18, 19, 20, or more (or any range derivable therein).

In certain aspects, the method comprises generating a set of topic genes for use in the topic modeling. The topic genes may be genes relevant to certain topics. The topic genes may be genes controlling certain topics. In some aspects, the set of topic genes comprises genes having a log fold change greater than 0.5. In some aspects, the set of topic genes comprises genes having a log fold change less than −0.5. The fold change may be in reference to a standard. The fold change may be in reference to an average amount, expression, or transcript reads of the cell population. The fold change may be in reference to a housekeeping gene. In some aspects, the set of topic genes comprises genes having a linear-feedback shift register less than 0.001.

In certain aspects, an RNA velocity parameter is calculated. The RNA velocity parameter may comprise an RNA velocity parameter estimation by a computational model. The RNA velocity parameter may comprise an RNA velocity parameter estimation by a one-state model. The RNA velocity parameter may comprise an RNA velocity parameter estimation by a geometric burst model.

Certain methods concern building a model to distinguish cell types in a plurality of cells. In some aspects, the method comprises receiving single cell RNA-sequencing (scRNA-seq) data from each cell in the plurality of cells. In some aspects, the method comprises fitting a topic model to a counts matrix from the scRNA-seq data to generate at least one topic. In some aspects, the counts matrix comprises spliced and unspliced transcript data. In certain aspects, the method comprises fitting a geometric burst model to the scRNA-seq data. In certain aspects, the burst model finds the probability of observing a cell with unspliced transcripts and spliced transcripts at a specific time.

In some aspects, the topic model comprises a set number of topics. In certain aspects, the set number of topics is a calculated optimal number of topics. In some aspects, the set number of topics is 1, 2, 3, 4, 5, 6, 7, 8, 9, 10, 11, 12, 13, 14, 15, 16, 17, 18, 19, or 20 topics. In some aspects, the method comprises integrating process-specific transition matrices by characterizing a probabilistic flow of topic-specific, which may be referred to as process-specific, transcriptional changes across a population of topic-associated cells, which may be referred to as process-associated cells. In some aspects, the spliced and unspliced transcript data comprises data from topic-specific genes, which, in some aspects, may be referred to as process-specific genes. The process-specific genes may be genes that are associated with specific cellular processes, such as transcription, translation, signaling, differentiation, and/or immune response. In some aspects, the topic-specific gene data is combined across the topic(s) and a global transition matrix is computed. In some aspects, the set of topic genes comprises genes having a log fold change greater than 0.5. In some aspects, the set of topic genes comprises genes having a log fold change less than −0.5. The fold change may be in reference to a standard. The fold change may be in reference to an average amount, expression, or transcript reads of the cell population. The fold change may be in reference to a housekeeping gene. In some aspects, the set of topic genes comprises genes having a linear-feedback shift register less than 0.001.

In certain aspects, the burst model uses the rate of a Poisson process governing a burst event. In certain aspects, the burst model uses the probability of producing the unspliced transcript during a single burst event governed by a geometric distribution. In certain aspects, the burst model uses a splicing rate of the unspliced transcripts. In certain aspects, the burst model uses a degradation rate of the spliced transcripts. In some aspects, at least one parameter of the geometric burst model is optimized by a Gillespie algorithm. The Gillespie algorithm may estimate a full joint distribution of the spliced and unspliced transcript counts. In some aspects, at least one parameter of the geometric burst model is optimized by a Nelder-Mead algorithm. The Nelder-Mead algorithm may estimate at least one maximum likelihood parameter value.

Certain methods concern determining cell fate in a plurality of cells. Certain methods concern analyzing a disease state from a plurality of cells. In some aspects, the methods determine cell types that are indicative of a disease, such as cell types corresponding to an immune response, an infection, or a cancer. Certain methods herein concern building a profile of a plurality of cells. Certain methods concern determining the efficacy of a treatment. In some aspects, the methods described herein build a profile of a plurality of cells before and after a treatment to determine the efficacy of the treatment. In some aspects, a change in the profile after the treatment can be used by one skilled in the art to determine the efficacy of the treatment. Certain methods concern determining the rate and/or direction of changes in transcriptional states. In some aspects, determining the rate and/or direction of change provides origins and destinations of transitioning cell states. Certain methods concern determining how a cell state specific to a disease state may arise via transformations of other cell states. Certain methods concern administering a therapeutic composition to a patient that has been determined to be in need of the therapeutic composition from the methods described herein. In some aspects, the method is used to determine how a treatment may affect one or more cells or a patient. In some aspects, the method is used to determine whether or not a treatment will affect one or more cells or a patient. Certain methods concern methods for managing treatment of a subject. In some aspects, the method comprises performing any of the computational methods described herein to generate a model and generating a treatment response output based on the model.

Certain aspects concern non-transitory storage media storing a set of instructions, that when executed, cause one or more computer processors to perform any of the methods described herein. Certain aspects concern non-transitory storage media storing a set of instructions, that when executed, cause one or more computer processors to build a model of cellular trajectory in a plurality of cells, the set of instructions comprising instructions to perform the steps of any of the methods described herein. Certain aspects concern non-transitory storage media storing a set of instructions, that when executed, cause one or more computer processors to build a model to distinguish cell types in a plurality of cells, the set of instructions comprising instructions to perform the steps of any of the methods described herein.

Certain aspects concern systems for performing any of the methods described herein. Certain methods concern systems for building a model of cellular trajectory in a plurality of cells. In some aspects, the system comprises a database storing single cell RNA-sequencing (scRNA-seq) data. In some aspects, the system comprises one or more computer processors operatively couple to said database wherein said one or more computer processors are individually or collectively programmed to perform the steps of any of the methods described herein. Certain aspects concern systems for building a model to distinguish cell types in a plurality of cells.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any aspect discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific aspects of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific aspects presented herein.

FIGS. 1A-1H show topic modeling enables RNA velocity inference to better capture local dynamics. FIG. 1A, UMAP embedding of scRNA-seq profiles of cells from COVID patient blood samples, colored by published annotations [38], shows RNA velocity transition streamlines (black arrows) reproduced using the scVelo dynamical model [5], which predict transdifferentiation of IgM plasmablasts (PBs) into developing neutrophils (DNs) (orange arrow). FIG. 1B, UMAP plots of cells colored by log-normalized expression of genes associated with the inferred PB-DN transition. FIG. 1C, For each of topics 3, 4, and 8 in an 11-topic model, (top) a UMAP shows cells colored by topic weights, and (bottom) a bar chart shows the log-fold change (x axis) and z-score (bar color) of the top 10 topic-specific genes (Methods). FIG. 1D, UMAP shows cells (in pink) used to produce the streamlines (black arrows) inferred from a combined analysis of topics 3, 4, and 8, which does not suggest trans-differentiation of PBs into DNs (orange arrows). FIGS. 1E-1F, Relative to a global analysis, topic-specific inference gives markedly different results for topic-3 specific gene PLBD1. (e) (left) Scatter plot shows phase portraits for either a global (red) or topic-specific (gold) fit; background shows all cells, colored by topic-3 weight, plotted by the number of spliced (s, x axis) versus unspliced (u, y axis) transcripts. (right) UMAP colored by the relative difference of inferred topic-specific and global velocity in topic-associated cells. (f) Cell-state transitions (arrows) inferred (left) globally, or (right) from topic-specific velocities, shown embedded in the phase plot, as in e. FIGS. 1G-1H, Analogous to e, f, for the topic-8 specific gene MPO.

FIGS. 2A-2D show TopicVelo combines topic modeling and a burst model for accurate, robust RNA velocity inference. FIG. 2A, Topic modeling of the joint spliced and unspliced count matrix reveals distinct gene programs associated with distinct functional responses or cell-fate decisions in a heterogenous population of cells. FIG. 2B, The transcriptional dynamics are inferred using a chemical master equation for a burst model, where β is the splicing rate, γ is the degradation rate, kₒₙ characterizes the typical waiting time for a burst event, and b is the burst size governed by a geometric distribution. The joint distribution of a typical gene over all cells is very concentrated at (0,0) because the gene is not involved in most cell states and transitions. By zooming in on cells associated with a specific topic, the joint distribution reveals detailed, process-specific dynamics of a topic-specific gene. Model parameters are estimated by minimizing the KL divergence between the inferred and experimentally observed joint distributions of the spliced and unspliced transcripts. FIG. 2C, TopicVelo uses the process-specific velocities to infer process-specific transition matrices, which it then integrates using topic weights into a global transition matrix. FIG. 2D, The results facilitate more robust, accurate downstream analyses, such as visualization of transition streamlines and trajectory inference.

FIGS. 3A-3I show TopicVelo infers differentiation trajectories of human hematopoiesis previously recovered only with the aid of metabolic labeling. FIG. 3A, Previously published UMAP embedding of human hematopoiesis data shows streamlines inferred with the aid of metabolically labeled transcripts over cells colored by cell-type annotation [20]; basophil (Bas), erythrocyte (Ery); granulocyte and monocyte progenitor (GMP-like); hematopoietic stem cell (HSC); megakaryocyte and erythrocyte progenitor (MEP-like); megakaryocyte (Meg), monocyte (Mon), and neutrophil (Neu). FIGS. 3B-3C, Same UMAP, but with streamlines produced without metabolic labels, using (b) the scVelo dynamical model, which, outside monocytes, fails to capture major differentiation trajectories (red arrows), or (c) TopicVelo with an 8-topic model, which correctly infers major differentiation trajectories (Mon, Bas, Ery, and Meg) (green arrows). FIGS. 3D-3E, Plots show the experimental joint distribution of spliced and unspliced mRNA counts in (d) all cells, or (e) topic-3 associated cells, of the topic-3 specific gene F13A1, which is known to be expressed in megakaryocytes [47]. FIGS. 3F-3G, Plots show the joint distribution of F13A1 in topic-3 associated cells, inferred using (f) the one-state model, or (g) maximum likelihood estimates for the burst model; the latter better captures both the diffuseness of the joint distribution and the relatively high probability at (0,0). FIGS. 3H-3I, UMAP close-ups show topic-3 associated cells (in blue) plotted under topic-3 specific streamlines, produced by (h) the scVelo dynamical model, or (i) TopicVelo.

FIGS. 4A-4H show TopicVelo correctly captures mouse erythropoiesis and human bone marrow development trajectories. FIG. 4A, Previously published UMAP embedding of cells involved in erythropoiesis, part of a larger single-cell mouse gastrulation dataset, with cells colored by cell-type annotation, shows streamlines (arrows) inferred by the scVelo stochastic model, which erroneously suggest differentiation of erythroid 3 into erythroid 2 cells (red arrow) [21, 23]. FIG. 4B, The same UMAP shows streamlines inferred by TopicVelo, which delineate the biologically expected differentiation trajectory (green arrow). FIG. 4C, UMAP plots for the gene Smim1 show cells colored by (left) size-normalized smoothed spliced mRNA counts, and by velocities (negative, red; positive, blue) inferred by (middle) scVelo, or by (right) TopicVelo. FIG. 4D, Analogous to c, for the gene Gata2. FIG. 4E, Previously published t-SNE plot of cells from human bone marrow, colored by annotated cell type [52], shows streamlines inferred by scVelo in stochastic mode, which predicts incorrectly that precursors, megakaryocytes, and erythrocytes differentiate into hematopoietic stem cells (red arrow). FIG. 4F, Same t-SNE plot shows streamlines inferred by TopicVelo, using 10 topics, which recovers the expected trajectories for all major lineages (green arrow). FIGS. 4G-4H, TopicVelo gives markedly different velocity results, compared to scVelo, for topic-specific genes. (g) For the erythroid-associated gene KLF1, UMAP plots colored by (left) smoothed normalized expression, and by velocities inferred by (middle) scVelo or (right) TopicVelo, which, in contrast to scVelo, captures the up-regulation of KLF1 in early erythroid development. (h) Analogous to g, for the monocyte-associated gene MPO.

FIGS. 5A-5I show TopicVelo correctly infers previously missed transitions in small cell populations during dentate gyrus and pancreas development. FIG. 5A, Previously published UMAP embedding of cells from dentate gyrus data, colored by cell-type annotation [5, 60], shows streamlines (black arrows) by scVelo in dynamical mode, which reveals major differentiation trajectories but not the transition from oligondendrocyte precursors (OPC) to oligodendrocytes (OLs) (orange box). FIG. 5B, Same UMAP shows streamlines from TopicVelo with 10 topics, which recovers the expected differentiation OPC-OL trajectory (green arrow), and the other major trajectories. FIG. 5C, For topic 2, (left) UMAP is colored by topic weights, and (right) a bar chart shows the topic-specific genes with largest log-fold change. FIG. 5D, Close-up on OL lineages of UMAPs, colored by (left two panels) smoothed size-normalized expression of topic-2 specific genes Ppp1r14 and Enpp2, and (right two panels) velocities for Enpp2 inferred by scVelo and TopicVelo. FIG. 5E, Previously published UMAP embedding of cells from pancreas endocrinogenesis data, colored by cell-type annotation [5, 61] shows streamlines (black arrows) by scVelo in dynamical mode, which predicts major differentiation trajectories but misses the pre-endocrine to δ cells transition (purple box, red arrow), and suggests a clear transition from pre-endocrine to β cells (black box). FIG. 5F, Same UMAP shows streamlines by TopicVelo with 6 topics, which correctly recovers all major transitions, including the commitment of δ cells (purple box, green arrow), and suggests uncertainty in the α-β branch point from pre-endocrine cells (black box). FIGS. 5G-5H, Analysis of topic 4, as in c, d, highlighting the topic-4 specific genes Sst, a δ marker, and the spliced and unspliced forms of Spock3 (Supplementary Table 1). FIGS. 5I-5G, Analogous to g, for topic 5; Chgb and Fev expression suggests cells not fully committed to α or β fates [66].

FIGS. 6A-6G show TopicVelo reveals complex transitions underlying the inflammatory response of skin ILCs with data from one of five time points. FIG. 6A, Previously published force-directed layout (FDL) embedding of scRNA-seq profiles of skin innate lymphoid cells (ILCs) from a mouse model of psoriasis [32] are colored in each panel by day of collection (panel title). The region of cells identified as ILC3-like (orange circle) contains no cells on day 0 but a sizable number of cells on day 3. FIG. 6B, FDL plots colored by pseudotime for transitions previously discovered using directed diffusion and topic modeling, with arrows indicating time point information. c-e Highlights from topic model of day 3 cells only. (FIG. 6C) For topic 4, the same FDL plot shows day 3 cells, colored by (top) topic-4 weights and (bottom) the log-scale expression 2 topic-4 specific genes; bar chart (right) shows topic-4 specific genes with largest log-fold change. (FIGS. 6D-6E) Analogous to c, for topics 5 and 9, respectively. FIGS. 6F-6G, RNA velocity analyses of topics 4, 6, and 9. FDL plots indicate the day 3 cells associated with topics 4, 6, and 9 (color), which were used to infer the streamlines (black arrows) by (left) scVelo in dynamical mode or by (right) TopicVelo. Focusing on the transitions to ILC3-like cells (orange oval), both predict the quiescent-to-ILC3 transition. However, scVelo erroneously suggests that ILC3s may change into ILC2 cells (red arrow), whereas TopicVelo correctly predicts an ILC2-to-ILC3 transition (green arrow).

FIGS. 7A-7I show original analysis from Wilk et. al argues plasmablasts transdifferentiate into developing neutrophils but topic modeling results generated from aspects herein question the robustness of this analysis. FIG. 7A, Original streamlines from Wilk et. al. [38] identifies a clear transdifferentiation from plasmablasts to developing neutrophils. This transition is highlighted by an orange rectangle. FIG. 7B, For topic 1, the top left shows the topic weights for cells. The bar plot includes the list of top topic genes ranked by the log-fold change and colored by the absolute value of the Z-scores. ‘U’ indicates the unspliced transcripts. Two examples of gene expression (size-normalized counts averaged over nearest neighbors) are also shown. FIGS. 7C-7I, Same as b but for topics 1, 2, 5, 6, 7, 9, and 10 respectively. The bar plot for topic 5 is not colored because only one gene qualifies as a topic gene.

FIGS. 8A-8J show parameter inference of geometric burst model is accurate and recovers experimental distributions more faithfully than the one-state model. FIG. 8A, An example joint distribution of the geometric burst model with kₒₙ=0.5, b=5, and γ=0.3 simulated with the Gillespie algorithm (top) and the joint distribution generated by the parameters from the maximum likelihood estimate (bottom). FIG. 8B, Log-scale KL divergence landscape of icon and b when γ is fixed at 0.3. The true value of icon and b is marked by the red cross. The optimization path is represented by a series of points where the end point is represented as a upward triangle. The points are connected by a yellow line. The colors of the points correspond to the inferred values of γ during the optimization. FIG. 8C, Log-scale KL divergence landscape and optimization path in the dimension of γ and b when kₒₙ was fixed at 0.5. FIG. 8D, The table contains parameters correspond to 27 combinations which were used to choose a suitable simulation steps and maximum iteration allowed in optimization for the inference. FIG. 8E, Inference performance for the 27 parameter combinations when the simulation steps were fixed and the maximum iterations of the Nelder-Mead optimizer was allowed to vary. For each maximum iterations (represented as different colors and different columns in the bar plot), 10 replicas of the 27 combinations were performed, and each point corresponds to the average KL divergence of the 27 combinations. FIG. 8F, Inference performance for the 27 parameter combinations when the maximum iterations were fixed and simulation steps were allowed to vary. FIG. 8G, The experimental joint distribution of Grin2b from the granule mature cells in the dentategyrus dataset [60] (left), the maximum likelihood estimations of the distribution of Grin2b using the one-state model (middle) and the geometric burst model (right). The pink dashed box zooms onto region where the probability mass function of the observed is most concentrated in; inference from the burst model matches the observation closely. The KL divergence on the log scale of each maximum likelihood estimation to the observed distribution is presented. The burst model performed more than 10 folds better than the one state model. FIG. 8H, The log-scale KL divergence landscape to the observed distributions of Grin2b in γ v.s. b (left) and kₒₙ v.s. and b (right). The red dots correspond to the end points of the optimizer. FIGS. 8I-8J, Similar to (FIGS. 8G-8H) but for Btbd9 from the granule mature cells in the dentategyrus dataset [60]. The burst model clearly outperforms the one-state model indicated by a significantly lower KL divergence value.

FIGS. 9A-9H show Topic modeling dissects the simultaneous dynamics of the scNT-seq data. FIGS. 9A-H, For each panel, the top shows the topic weights for cells. The bottom left bar plot includes the list of top topic genes ranked by the log-fold change and colored by the absolute values of the Z-scores. ‘U’ indicates the unspliced transcripts. Two examples of gene expression (log-scale of the size-normalized count) are shown on the bottom right.

FIGS. 10A-10E show TopicVelo recovers more biologically plausible velocity comparing to scVelo for the scNT-seq data. FIGS. 10A-10E, For each gene (row), the smoothed unspliced, smoothed spliced expression, the velocities inferred by scVelo and TopicVelo are shown from left to right.

FIGS. 11A-11B show Topic modeling of the gastrulation data revels key genes underlying the differentiation of blood progenitors to erythroid. FIG. 11A, For topic 0 which is strongly associated with the later stage of erythroid, the top shows the topic weights for cells. The bottom left bar plot includes the list of top topic genes ranked by the log-fold change and colored by the absolute values of the Z-scores. ‘U’ indicates the unspliced transcripts. Examples of gene expression (log-scale of the size-normalized count) are shown on the bottom right. FIG. 11B, Same as FIG. 11A but for topic 1 which is correlated with the blood progenitors and early stages of erythroid.

FIGS. 12A-12B show TopicVelo recovers more biologically accurate velocity than scVelo for the gastrulation data. FIGS. 12A-12B, For each gene (row), the smoothed unspliced, smoothed spliced expression, the velocities inferred by scVelo and TopicVelo are shown from left to right.

FIGS. 13A-13J show topic modeling of the human bone marrow data provides insights into the different stages of differentiation along all lineages. FIG. 13A, For topic 0 which is associated with matured erythroid, the top shows the topic weights for cells. The bar plot in the middle includes the list of top topic genes ranked by the log-fold change and color by the absolute values of the Z-scores. ‘U’ indicates the unspliced transcripts. At the bottom, examples of gene expression (log-scale of the size-normalized count) are also shown. FIGS. 13B-13J, Same as FIG. 13A but topics 1 (megakaryocyte), 2 (dendritic cells), 3 (one lineage of monocytes), 4 (common lymphoid progenitors), 5 (hematopoietic stem cells), 6 (erythroid), 7 (another lineage for monocytes), 8 (precursors to monocytes), 9 (hematopoietic stem cells and precursors to dendritic cells).

FIGS. 14A-14E show TopicVelo recovers more biologically supported velocity than scVelo for the human bone marrow data. FIGS. 14A-14E, For each gene (row), the smoothed unspliced, smoothed spliced expression, the velocities inferred by scVelo and TopicVelo are shown from left to right.

FIGS. 15A-15I show additional topic modeling results of the dentate gyrus dataset reveals dynamical changes for both of the astrocytes and granule lineages in addition to identify key genes of rare cell types. FIG. 15A, Topic 0 is strong associated with the radial glia-like cells and astrocytes. The top left bar chart shows the topic genes with the highest log-fold changes colored by their absolute values of Z-scores. ‘U’ indicates the unspliced transcripts. The middle shows the topic weights on a UMAP embedding. The bottom left and right are the size-normalized expression of key genes on the log-scale. FIG. 15B, Same for topic 1 which is a proliferation programs in which the topic genes are highly expressed across majority of cells. FIG. 15C, Same for topic 3 which is strongly associated with CCK, GABA and CR cells. FIG. 15D, Same for topic 4 which is strongly associated with microglia and endothelial cells. FIG. 15E, Same for topic 5 which is related to neuroblasts and early granule immature cells. FIGS. 15F-15G, Same for topics 6 and 7 a which are connected to the granule lineage. FIG. 15H, Same for topic 4 which is strongly associated with mossy cells. FIG. 15I, Same for topic 9 which is related to neuroblasts and radial glia-like cells.

FIGS. 16A-16D show additional topic modeling results of the pancreas dataset provide a comprehensive portrayal of the cell cycles of ductal cells, transience of intermediate stage in addition to maturation of ϵ and β cells. FIG. 16A, Topic 0 is strongly associated with ϵ cells. Top left is the topic weights on a UMAP embedding. Top right presents a bar chart for topic genes with the highest log-fold changes colored by their absolute values of Z-scores. ‘U’ indicates the unspliced transcripts. The bottom left and right are the size-normalized expression of key genes on the log-scale. FIG. 16B, Same for topic 1 which is associated with the cell cycle of ductal cells. FIG. 16C, Same for topic 2 which is associated with Ng3n high EP and Ng3n low EP cells. FIG. 16D, Same for topic 3 which is associated with β cells.

FIGS. 17A-17G show topic modeling results for the ILC data from only day 3. FIG. 17A, For topic 0, the top left shows the topic weights for cells on a force-directed layout embedding. The bottom left bar plot includes the list of top topic genes ranked by the log-fold changes and colored by the absolute values of the Z-scores. ‘U’ indicates the unspliced transcripts. Examples of gene expression (log-scale of the size-normalized count) are shown on the bottom right. FIGS. 17B-17G, Same as FIG. 17A but for topics 1, 2, 3, 5, 7, and 8 respectively.

FIGS. 18A-18E show TopicVelo recovers more biologically plausible velocity than scVelo for the ILC data. FIGS. 18A-18E, For each gene (row), the smoothed unspliced, smoothed spliced expression, the velocities inferred by scVelo and TopicVelo are shown from left to right.

### DETAILED DESCRIPTION OF THE INVENTION

A key challenge in transcriptomic data science, trajectory inference (TI) estimates the positions of individual cells in an underlying, ordered biological process from destructively measured single-cell RNA-sequencing profiles. Previous works presented a time derivative of the transcriptional state, termed RNA velocity, which was estimated by fitting unspliced and spliced mRNA counts to simple kinetic models. While RNA velocity has the potential power to reveal flow patterns of transcriptional changes, even within one time point, it lacks robustness and accuracy, particularly in applications beyond highly structured, primarily developmental, contexts. Aspects herein show that global inference of RNA velocity can be distorting. To apply RNA velocity to more general systems, including immune response studies, aspects herein concern a new approach that can infer the cells and genes associated with distinct active processes via probabilistic topic modeling and uses the results to estimate process-specific velocity parameters. In some aspects, process-specific transition weights are then integrated to estimate large-scale transition matrices. In some aspects, parameter accuracy is also improved by efficiently fitting unsmoothed counts to a transcriptional burst model. In varied datasets, certain aspects outperformed the known methods, recovering parameters and transition flows that were experimentally supported or previously recovered only with the aid of metabolic labeling or multiple time points.

Aspects herein create an RNA velocity tool that can be effectively applied to investigate general systems, including immune responses, which may be referred to as TopicVelo. The method provides, in some aspects, a new approach that reflects the finding that, in complex systems, it is critical to focus parameter estimation for a dynamic process on relevant cells and genes. To disentangle potentially simultaneous dynamical processes, in some aspects, TopicVelo utilizes probabilistic topic modeling [27, 28], a Bayesian form of non-negative matrix factorization [29], also known as admixture [30] and/or grade-of-membership modeling [31]. In certain aspects, topic modeling assigns each cell a set of “topic” weights, which may represent the relative activity levels of the jointly inferred “topics” or processes, each may be represented by a probability distribution across genes. In some aspects, the model allows continuous transcriptional heterogeneity to be captured and strikes a useful balance between scRNA− seq data compression and biological interpretability (e.g., [32-35]). From process-associated cells and genes, in some aspects, TopicVelo infers process-specific velocity parameters and transition matrices, which, in some aspects, TopicVelo then integrates, using the topic weights, to estimate meaningful, large-scale transitions that may involve multiple processes.

In some aspects, to infer gene-specific kinetic parameters, TopicVelo fits integer counts to a physically meaningful transcription model, rather than performing the standard smoothing of spliced and unspliced counts over cell neighborhoods in a k-nearest-neighbors (k-NN) graph computed from the top principal components of global gene expression. Nearly all previous RNA velocity methods [4, 5, 15, 17, 20] first smooth counts and then fit the data using ordinary differential equations (ODEs). In contrast, in certain aspects, TopicVelo efficiently approximates the full joint distributions of unsmoothed spliced and unspliced counts based on a chemical master equation for the geometric burst model [36]. Consistent with previous arguments [22, 24, 37], aspects herein show that using unsmoothed counts and a burst model substantially improve kinetic parameter estimates in the applications.

Aspects herein assess the performance of TopicVelo across diverse datasets, varying in organism, tissue, and biological focus. In all cases, TopicVelo performed significantly better than a previous approach, scVelo [5]. Specifically, aspects recovered velocities and transition flows more consistent with observed expression patterns and experimental validations, without elaborate parameter tuning. In certain aspects herein, TopicVelo infers dynamics whose inference previously depended on the aid of metabolic labeling [20] or multiple time points [32].

### I. Data Acquisition

A. RNA Sequencing

Input data for the methods described herein may comprise raw sequencing reads of RNA from subjects (e.g., patients), including raw sequencing reads from individual cells. In some aspects, RNA may be analyzed by sequencing. The RNA may be prepared for sequencing by any method known in the art, such as poly-A selection, cDNA synthesis, stranded or nonstranded library preparation, or a combination thereof. The RNA may be prepared for any type of RNA sequencing technique, including stranded specific RNA sequencing. In some aspects, sequencing may be performed to generate approximately 10M, 15M, 20M, 25M, 30M, 35M, 40M or more reads, including paired reads. The sequencing may be performed at a read length of approximately 50 bp, 55 bp, 60 bp, 65 bp, 70 bp, 75 bp, 80 bp, 85 bp, 90 bp, 95 bp, 100 bp, 105 bp, 110 bp, or longer. In some aspects, raw sequencing data may be converted to estimated read counts (RSEM), fragments per kilobase of transcript per million mapped reads (FPKM), and/or reads per kilobase of transcript per million mapped reads (RPKM). In some aspects, one or more bioinformatics tools may be used to infer stroma content, immune infiltration, and/or tumor immune cell profiles, such as by using upper quartile normalized RSEM data.

B. Single-Cell Analysis Techniques

1. Drop-Seq

Drop-Seq analyzes mRNA transcripts from droplets of individual cells in a highly parallel fashion. This single-cell sequencing method uses a microfluidic device to compartmentalize droplets containing a single cell, lysis buffer, and a microbead covered with barcoded primers. Each primer contains: 1) a 30 bp oligo(dT) sequence to bind mRNAs; 2) an 8 bp molecular index to identify each mRNA strand uniquely; 3) a 12 bp barcode unique to each cell and 4) a universal sequence identical across all beads. Following compartmentalization, cells in the droplets are lysed and the released mRNA hybridizes to the oligo(dT) tract of the primer beads. Next, all droplets are pooled and broken to release the beads within. After the beads are isolated, they are reverse-transcribed with template switching. This generates the first cDNA strand with a PCR primer sequence in place of the universal sequence. cDNAs are PCR-amplified, and sequencing adapters are added using the Nextera XT Library Preparation Kit. The barcoded mRNA samples are ready for sequencing. This method is further described in Macosko, Evan Z., et al., Cell, 2015. 161(5): p. 1202-1214, which is herein incorporated by reference.

2. inDrop

inDrop is used for high-throughput single-cell labeling. This approach is similar to Drop-seq, but it uses hydrogel microspheres to introduce the oligonucleotides. Single cells from a cell suspension are isolated into droplets containing lysis buffer. After cell lysis, cell droplets are fused with a hydrogel microsphere containing cell-specific barcodes and another droplet with enzymes for RT. Droplets from all the wells are pooled and subjected to isothermal reactions for RT. The barcodes anneal to poly(A)+ mRNAs and act as primers for reverse transcriptase. Now that each mRNA strand has cell-specific barcodes, the droplets are pooled and broken, and the cDNA is purified. The 3′ ends of the cDNA strands are ligated to adapters, amplified, annealed to indexed primers, and amplified further before sequencing. This method is further described in Klein, Allon M., et al., Cell, 2015. 161(5): p. 1187-1201, which is herein incorporated by reference.

3. CEL-Seq

CEL-Seq uses barcoding and pooling of RNA to overcome challenges from low input. In this method, each cell undergoes RT with a unique barcoded primer in its individual tube. After second-strand synthesis, cDNAs from all reaction tubes are pooled and PCR-amplified. Paired-end deep sequencing of the PCR products allows for accurate detection of sequence information derived from both strands. This method, and related CEL-seq2 are further described in Hashimshony, T., et al., Cell Reports, 2012. 2(3): p. 666-673 and Hashimshony, T., et al., Genome Biology, 2016. 17(1): p. 77, which are herein incorporated by reference.

4. Quartz-Seq

The Quartz-Seq method optimizes whole-transcript amplification (WTA) of single cells. In this method, an RT primer with a T7 promoter and PCR target is first added to the extracted mRNA. RT synthesizes first-strand cDNA, after which the RT primer is digested by exonuclease I. Next, a poly(A) tail is added to the 3′ ends of first-strand cDNA, along with a poly(dT) primer containing a PCR target. After second-strand generation, a blocking primer is added to ensure PCR enrichment in sufficient quantity for sequencing. Deep sequencing allows for accurate, high-resolution representation of the whole transcriptome of a single cell.

5. MARS-Seq

MARS-Seq profiles the transcriptional dynamics of single cells in an automated and massively parallel workflow with high resolution. MARS-Seq can be used with in vivo samples containing a wide variety of different cell subpopulations. Single cells are first isolated into individual wells using FACS. Each cell is lysed, and the 3′ ends of mRNAs are annealed to unique molecular identifiers containing a T7 promoter. The mRNA is reverse-transcribed to generate the first cDNA strand and treated with exonuclease I to remove leftover RT primers. Next, the cellular lysates are pooled together and converted to double-stranded cDNA. The DNA strands are transcribed to RNA and treated with DNase to remove leftover DNA templates in the mixture. The RNA strands are fragmented and annealed to sequencing adapters, followed by RT to generate barcoded cDNA libraries that are ready for sequencing.

6. CytoSeq

CytoSeq enables gene expression profiling of thousands of single cells. In this method, single cells are randomly deposited into wells. A combinatorial library of beads with specific capture probes is added to each well. After cell lysis, mRNAs hybridize the to beads, which are pooled subsequently for RT, amplification, and sequencing. Deep sequencing provides accurate, high-coverage gene expression profiles of several single cells.

7. Hi-SCL

Hi-SCL generates transcriptome profiles for thousands of single cells using a custom microfluidics system, similar to Drop-Seq and inDrop. Single cells from cell suspension are isolated into droplets containing lysis buffer. After cell lysis, cell droplets are fused with a droplet containing cell-specific barcodes and another droplet with enzymes for RT. The droplets from all the wells are pooled and subjected to isothermal reactions for RT. The barcodes anneal to poly(A)+ mRNAs and act as primers for reverse transcriptase. Now that each mRNA strand has cell-specific barcodes, the droplets are broken, and the cDNA is purified. The 3′ ends of the cDNA strands are ligated to adapters, amplified, annealed to indexed primers, and amplified further before sequencing.

8. Seq-Well

Single-cell RNA-seq can precisely resolve cellular states, but applying this method to low-input samples is challenging. Here, the inventors present Seq-Well, a portable, low-cost platform for massively parallel single-cell RNA-seq. Barcoded mRNA capture beads and single cells are sealed in an array of subnanoliter wells using a semipermeable membrane, enabling efficient cell lysis and transcript capture. This method is further described in Gierahn et al., Nat Methods. 2017 April; 14(4):395-398, which is herein incorporated by reference. This method is further described in Gierahn, T. M., et al., Nature Methods, 2017. 14: p. 395, which is herein incorporated by reference.

9. Microwell-Seq

Microwell-seq confines single cells and barcoded poly(dT) mRNA capture beads in a PDMS array of subnanoliter wells. Well dimensions are designed to accommodate only one bead. Cells are loaded by gravity with a rate of dual occupancy that can be tuned by adjusting the number of cells and loaded and visualized prior to processing. This method is further described in Han, X., et al., Cell, 2018. 172(5): p. 1091-1107.e17, which is herein incorporated by reference.

10. Nanogrid-Seq

Nanogrid-seq is a nanogrid platform and microfluidic depositing system that enables imaging, selection, and sequencing of thousands of single cells or nuclei in parallel. This method is further described in Gao, R., et al., Nature Communications, 2017. 8(1): p. 228, which is herein incorporated by reference.

11. Sci-Seq

Sci-seq refers to Single cell Combinatorial Indexed Sequencing (SCI-seq) that can be used as a means of simultaneously generating thousands of low-pass single cell libraries for somatic copy number variant detection. This is further described in Vitak, S. A., et al., Nature Methods, 2017. 14: p. 302, which is herein incorporated by reference.

12. Direct-Tagmentation

Enzymes called transposases randomly cut the DNA into short segments (“tags”). Adapters are added on either side of the cut points (ligation). Strands that fail to have adapters ligated are washed away. The adaptors may contain barcodes and/or primer binding sites for detection and amplification of the genomic sequences. This is further described in Zahn, H., et al., Nature Methods, 2017. 14: p. 167, which is herein incorporated by reference.

13. sciATAC-Seq

sci-ATAC-seq is a single-cell ATAC-seq protocol. This technique can be used to determine chromatin accessibility both between and within populations of single cells. Single-cell ATAC-Seq relies on combinatorial cellular indexing, and thus does not require the physical isolation of individual cells during library construction. The technique scales sublinearly in time and cost and can profile thousands of individual cells in a single experiment. This method is further described in Cusanovich, D. A., et al., Science, 2015. 348(6237): p. 910, which is herein incorporated by reference. A related method, nano-well scATAC-seq is described in Mezger, A., et al., High-throughput chromatin accessibility profiling at single-cell resolution, bioRxiv, 2018, which is incorporated by reference.

Other methods include 10× genomics RNA sequencing platform, described in Zheng, G. X. Y., et al., Nature Communications, 2017. 8: p. 14049; SMART-seq, described in Ramskold, D., et al., Nature Biotechnology, 2012. 30: p. 777; SMART-seq2, described in Picelli, S., et al., Nature Protocols, 2014. 9: p. 171, which are all herein incorporated by reference in their entirety. It is contemplated that aspects in the disclosed references may be incorporated into aspects described in this disclosure.

C. Sequencing Methods

1. Massively Parallel Signature Sequencing (MPSS).

The first of the next-generation sequencing technologies, massively parallel signature sequencing (or MPSS), was developed in the 1990s at Lynx Therapeutics. MPSS was a bead-based method that used a complex approach of adapter ligation followed by adapter decoding, reading the sequence in increments of four nucleotides. This method made it susceptible to sequence-specific bias or loss of specific sequences. Because the technology was so complex, MPSS was only performed ‘in-house’ by Lynx Therapeutics and no DNA sequencing machines were sold to independent laboratories. Lynx Therapeutics merged with Solexa (later acquired by Illumina) in 2004, leading to the development of sequencing-by-synthesis, a simpler approach acquired from Manteia Predictive Medicine, which rendered MPSS obsolete. However, the essential properties of the MPSS output were typical of later “next-generation” data types, including hundreds of thousands of short DNA sequences. In the case of MPSS, these were typically used for sequencing cDNA for measurements of gene expression levels. Indeed, the powerful Illumina HiSeq2000, HiSeq2500 and MiSeq systems are based on MPSS.

2. Polony Sequencing.

The Polony sequencing method, developed in the laboratory of George M. Church at Harvard, was among the first next-generation sequencing systems and was used to sequence a full genome in 2005. It combined an in vitro paired-tag library with emulsion PCR, an automated microscope, and ligation-based sequencing chemistry to sequence an E. coli genome at an accuracy of >99.9999% and a cost approximately 1/9 that of Sanger sequencing. The technology was licensed to Agencourt Biosciences, subsequently spun out into Agencourt Personal Genomics, and eventually incorporated into the Applied Biosystems SOLiD platform, which is now owned by Life Technologies.

3. 454 Pyrosequencing.

A parallelized version of pyrosequencing was developed by 454 Life Sciences, which has since been acquired by Roche Diagnostics. The method amplifies DNA inside water droplets in an oil solution (emulsion PCR), with each droplet containing a single DNA template attached to a single primer-coated bead that then forms a clonal colony. The sequencing machine contains many picoliter-volume wells each containing a single bead and sequencing enzymes. Pyrosequencing uses luciferase to generate light for detection of the individual nucleotides added to the nascent DNA, and the combined data are used to generate sequence read-outs. This technology provides intermediate read length and price per base compared to Sanger sequencing on one end and Solexa and SOLiD on the other.

4. Illumina (Solexa) Sequencing.

Solexa, now part of Illumina, developed a sequencing method based on reversible dye-terminators technology, and engineered polymerases, that it developed internally. The terminated chemistry was developed internally at Solexa and the concept of the Solexa system was invented by Balasubramanian and Klennerman from Cambridge University's chemistry department. In 2004, Solexa acquired the company Manteia Predictive Medicine in order to gain a massively parallel sequencing technology based on “DNA Clusters”, which involves the clonal amplification of DNA on a surface. The cluster technology was co-acquired with Lynx Therapeutics of California. Solexa Ltd. later merged with Lynx to form Solexa Inc.

In this method, DNA molecules and primers are first attached on a slide and amplified with polymerase so that local clonal DNA colonies, later coined “DNA clusters”, are formed. To determine the sequence, four types of reversible terminator bases (RT-bases) are added and non-incorporated nucleotides are washed away. A camera takes images of the fluorescently labeled nucleotides, then the dye, along with the terminal 3′ blocker, is chemically removed from the DNA, allowing for the next cycle to begin. Unlike pyrosequencing, the DNA chains are extended one nucleotide at a time and image acquisition can be performed at a delayed moment, allowing for very large arrays of DNA colonies to be captured by sequential images taken from a single camera.

Decoupling the enzymatic reaction and the image capture allows for optimal throughput and theoretically unlimited sequencing capacity. With an optimal configuration, the ultimately reachable instrument throughput is thus dictated solely by the analog-to-digital conversion rate of the camera, multiplied by the number of cameras and divided by the number of pixels per DNA colony required for visualizing them optimally (approximately 10 pixels/colony). In 2012, with cameras operating at more than 10 MHz A/D conversion rates and available optics, fluidics and enzymatics, throughput can be multiples of 1 million nucleotides/second, corresponding roughly to one human genome equivalent at 1× coverage per hour per instrument, and one human genome re-sequenced (at approx. 30×) per day per instrument (equipped with a single camera).

5. Solid Sequencing.

Applied Biosystems' (now a Thermo Fisher Scientific brand) SOLiD technology employs sequencing by ligation. Here, a pool of all possible oligonucleotides of a fixed length are labeled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting beads, each containing single copies of the same DNA molecule, are deposited on a glass slide. The result is sequences of quantities and lengths comparable to Illumina sequencing. This sequencing by ligation method has been reported to have some issue sequencing palindromic sequences.

6. Ion Torrent Semiconductor Sequencing.

Ion Torrent Systems Inc. (now owned by Thermo Fisher Scientific) developed a system based on using standard sequencing chemistry, but with a novel, semiconductor based detection system. This method of sequencing is based on the detection of hydrogen ions that are released during the polymerization of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

7. DNA Nanoball Sequencing.

DNA nanoball sequencing is a type of high throughput sequencing technology used to determine the entire genomic sequence of an organism. The company Complete Genomics uses this technology to sequence samples submitted by independent researchers. The method uses rolling circle replication to amplify small fragments of genomic DNA into DNA nanoballs. Unchained sequencing by ligation is then used to determine the nucleotide sequence. This method of DNA sequencing allows large numbers of DNA nanoballs to be sequenced per run and at low reagent costs compared to other next generation sequencing platforms. However, only short sequences of DNA are determined from each DNA nanoball which makes mapping the short reads to a reference genome difficult. This technology has been used for multiple genome sequencing projects.

8. Heliscope Single Molecule Sequencing.

Heliscope sequencing is a method of single-molecule sequencing developed by Helicos Biosciences. It uses DNA fragments with added poly-A tail adapters which are attached to the flow cell surface. The next steps involve extension-based sequencing with cyclic washes of the flow cell with fluorescently labeled nucleotides (one nucleotide type at a time, as with the Sanger method). The reads are performed by the Heliscope sequencer. The reads are short, up to 55 bases per run, but recent improvements allow for more accurate reads of stretches of one type of nucleotides. This sequencing method and equipment were used to sequence the genome of the M13 bacteriophage.

9. Single Molecule Real Time (SMRT) Sequencing.

SMRT sequencing is based on the sequencing by synthesis approach. The DNA is synthesized in zero-mode wave-guides (ZMWs)—small well-like containers with the capturing tools located at the bottom of the well. The sequencing is performed with use of unmodified polymerase (attached to the ZMW bottom) and fluorescently labelled nucleotides flowing freely in the solution. The wells are constructed in a way that only the fluorescence occurring by the bottom of the well is detected. The fluorescent label is detached from the nucleotide at its incorporation into the DNA strand, leaving an unmodified DNA strand. According to Pacific Biosciences, the SMRT technology developer, this methodology allows detection of nucleotide modifications (such as cytosine methylation). This happens through the observation of polymerase kinetics. This approach allows reads of 20,000 nucleotides or more, with average read lengths of 5 kilobases.]

D. Additional Assay Methods

In some aspects, arrays can be used to detect nucleic acids of the disclosure. An array comprises a solid support with nucleic acid probes attached to the support. Arrays typically comprise a plurality of different nucleic acid probes that are coupled to a surface of a substrate in different, known locations. These arrays, also described as “microarrays” or colloquially “chips” have been generally described in the art, for example, U.S. Pat. Nos. 5,143,854, 5,445,934, 5,744,305, 5,677,195, 6,040,193, 5,424,186 and Fodor et al., 1991), each of which is incorporated by reference in its entirety for all purposes. Techniques for the synthesis of these arrays using mechanical synthesis methods are described in, e.g., U.S. Pat. No. 5,384,261, incorporated herein by reference in its entirety for all purposes. Although a planar array surface is used in certain aspects, the array may be fabricated on a surface of virtually any shape or even a multiplicity of surfaces. Arrays may be nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, which are hereby incorporated in their entirety for all purposes.

In addition to the use of arrays and microarrays, it is contemplated that a number of difference assays could be employed to analyze nucleic acids. Such assays include, but are not limited to, nucleic amplification, polymerase chain reaction, quantitative PCR, RT-PCR, in situ hybridization, digital PCR, dd PCR (digital droplet PCR), nCounter (nanoString), BEAMing (Beads, Emulsions, Amplifications, and Magnetics) (Inostics), ARMS (Amplification Refractory Mutation Systems), TAm-Seg (Tagged-Amplicon deep sequencing), PAP (Pyrophosphorolysis-activation polymerization), northern hybridization, hybridization protection assay (HPA)(GenProbe), branched DNA (bDNA) assay (Chiron), rolling circle amplification (RCA), single molecule hybridization detection (US Genomics), Invader assay (ThirdWave Technologies), and/or Bridge Litigation Assay (Genaco).

The use of a probe or primer of between 13 and 100 nucleotides, particularly between 17 and 100 nucleotides in length, or in some aspects up to 1-2 kilobases or more in length, allows the formation of a duplex molecule that is both stable and selective. Molecules having complementary sequences over contiguous stretches greater than 20 bases in length may be used to increase stability and/or selectivity of the hybrid molecules obtained. One may design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In one aspect, each probe/primer comprises at least 15 nucleotides. For instance, each probe can comprise at least or at most 20, 25, 50, 75, 100, 125, 150, 175, 200, 225, 250, 275, 300, 325, 350, 400 or more nucleotides (or any range derivable therein). They may have these lengths and have a sequence that is identical or complementary to a gene described herein. Particularly, each probe/primer has relatively high sequence complexity and does not have any ambiguous residue (undetermined “n” residues). The probes/primers can hybridize to the target gene, including its RNA transcripts, under stringent or highly stringent conditions. It is contemplated that probes or primers may have inosine or other design implementations that accommodate recognition of more than one human sequence for a particular biomarker.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

In one aspect, quantitative RT-PCR (such as TaqMan, ABI) is used for detecting and comparing the levels or abundance of nucleic acids in samples. The concentration of the target DNA in the linear portion of the PCR process is proportional to the starting concentration of the target before the PCR was begun. By determining the concentration of the PCR products of the target DNA in PCR reactions that have completed the same number of cycles and are in their linear ranges, it is possible to determine the relative concentrations of the specific target sequence in the original DNA mixture. This direct proportionality between the concentration of the PCR products and the relative abundances in the starting material is true in the linear range portion of the PCR reaction. The final concentration of the target DNA in the plateau portion of the curve is determined by the availability of reagents in the reaction mix and is independent of the original concentration of target DNA. Therefore, the sampling and quantifying of the amplified PCR products may be carried out when the PCR reactions are in the linear portion of their curves. In addition, relative concentrations of the amplifiable DNAs may be normalized to some independent standard/control, which may be based on either internally existing DNA species or externally introduced DNA species. The abundance of a particular DNA species may also be determined relative to the average abundance of all DNA species in the sample.

In one aspect, the PCR amplification utilizes one or more internal PCR standards. The internal standard may be an abundant housekeeping gene in the cell or it can specifically be GAPDH, GUSB and β-2 microglobulin. These standards may be used to normalize expression levels so that the expression levels of different gene products can be compared directly. A person of ordinary skill in the art would know how to use an internal standard to normalize expression levels.

A problem inherent in some samples is that they are of variable quantity and/or quality. This problem can be overcome if the RT-PCR is performed as a relative quantitative RT-PCR with an internal standard in which the internal standard is an amplifiable DNA fragment that is similar or larger than the target DNA fragment and in which the abundance of the DNA representing the internal standard is roughly 5-100 fold higher than the DNA representing the target nucleic acid region.

In another aspect, the relative quantitative RT-PCR uses an external standard protocol. Under this protocol, the PCR products are sampled in the linear portion of their amplification curves. The number of PCR cycles that are optimal for sampling can be empirically determined for each target DNA fragment. In addition, the nucleic acids isolated from the various samples can be normalized for equal concentrations of amplifiable DNAs.

A nucleic acid array can comprise at least 1, 2, 3, 4, 5, 6, 7, 8, 9, 10, 15, 20, 25, 30, 35, 40, 45, 50, 60, 70, 80, 90, 100, 150, 200, 250 or more different polynucleotide probes, which may hybridize to different and/or the same biomarkers. Multiple probes for the same gene can be used on a single nucleic acid array. Probes for other disease genes can also be included in the nucleic acid array. The probe density on the array can be in any range. In some aspects, the density may be or may be at least 50, 100, 200, 300, 400, 500 or more probes/cm2 (or any range derivable therein).

Certain aspects may involve the use of arrays or data generated from an array. Data may be readily available. Moreover, an array may be prepared in order to generate data that may then be used in correlation studies.

### II. Detection Kits and Systems

One can recognize that based on the methods described herein, detection reagents, kits, and/or systems can be utilized to detect the RNA and/or perform the RNA-sequencing. The reagents can be combined into at least one of the established formats for kits and/or systems as known in the art. As used herein, the terms “kits” and “systems” refer to aspects such as combinations of at least one RNA detection reagent, for example at least one selective oligonucleotide probe, and at least one The kits could also contain other reagents, chemicals, buffers, enzymes, packages, containers, electronic hardware components, etc. The kits/systems could also contain packaged sets of primers, oligonucleotides, arrays, beads, barcodes or other detection reagents. Any number of probes could be implemented for a detection array. In some aspects, the detection reagents and/or the kits/systems are paired with chemiluminescent or fluorescent detection reagents. Particular aspects of kits/systems include the use of electronic hardware components, such as DNA chips or arrays, or microfluidic systems, for example. In specific aspects, the kit also comprises one or more therapeutic or prophylactic interventions in the event the individual is determined to be in need of.

### III. Formulations and Culture of the Cells

Certain aspects relate to the analysis of one or more cells, which may be in a cell population. The cells may be obtained from a subject. The cells may be any cell type including progenitor cells, immune cells, and/or neoplastic cells. The cells may be cultured prior to the analysis. In particular aspects, cells may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (Gibco), and Glutamax (Gibco).

In certain aspects, the medium may comprise one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific aspects, one or more of these may be explicitly excluded.

In some aspects, the medium further comprises vitamins. In some aspects, the medium comprises 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B 12, or the medium includes combinations thereof or salts thereof. In some aspects, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B 12. In some aspects, the vitamins include or consist essentially of biotin, DL alpha tocopherol acetate, DL alpha-tocopherol, vitamin A, or combinations or salts thereof. In some aspects, the medium further comprises proteins. In some aspects, the proteins comprise albumin or bovine serum albumin, a fraction of BSA, catalase, insulin, transferrin, superoxide dismutase, or combinations thereof. In some aspects, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some aspects, the medium comprises one or more of the following: a B-27® supplement, xeno-free B-27® supplement, GS21™ supplement, or combinations thereof. In some aspects, the medium comprises or further comprises amino acids, monosaccharides, inorganic ions. In some aspects, the amino acids comprise arginine, cystine, isoleucine, leucine, lysine, methionine, glutamine, phenylalanine, threonine, tryptophan, histidine, tyrosine, or valine, or combinations thereof. In some aspects, the inorganic ions comprise sodium, potassium, calcium, magnesium, nitrogen, or phosphorus, or combinations or salts thereof. In some aspects, the medium further comprises one or more of the following: molybdenum, vanadium, iron, zinc, selenium, copper, or manganese, or combinations thereof. In certain aspects, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21™ supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific aspects, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts. In specific aspects, one or more of these may be explicitly excluded.

One or more of the medium components may be added at a concentration of at least, at most, or about 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific aspects, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular aspects the cells are in a formulated cell suspension that is stable at room temperature for 1, 2, 3, or 4 hours or more from time of thawing.

### EXAMPLES

The following examples are included to demonstrate preferred aspects of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the invention.

### Example 1: Process-Specific Versus Global Inference of RNA Velocity

Standard estimation of RNA velocity parameters is based on a set of cells and genes globally determined to be in steady state. The inventors hypothesized that global inference may lead to distortions in estimated kinetic parameters for a local dynamic process, partly because cells and genes that are not associated with the process can still strongly influence parameter estimates, while key cells and genes involved in the process may poorly fit global models and hence be excluded from contributing to parameter estimation.

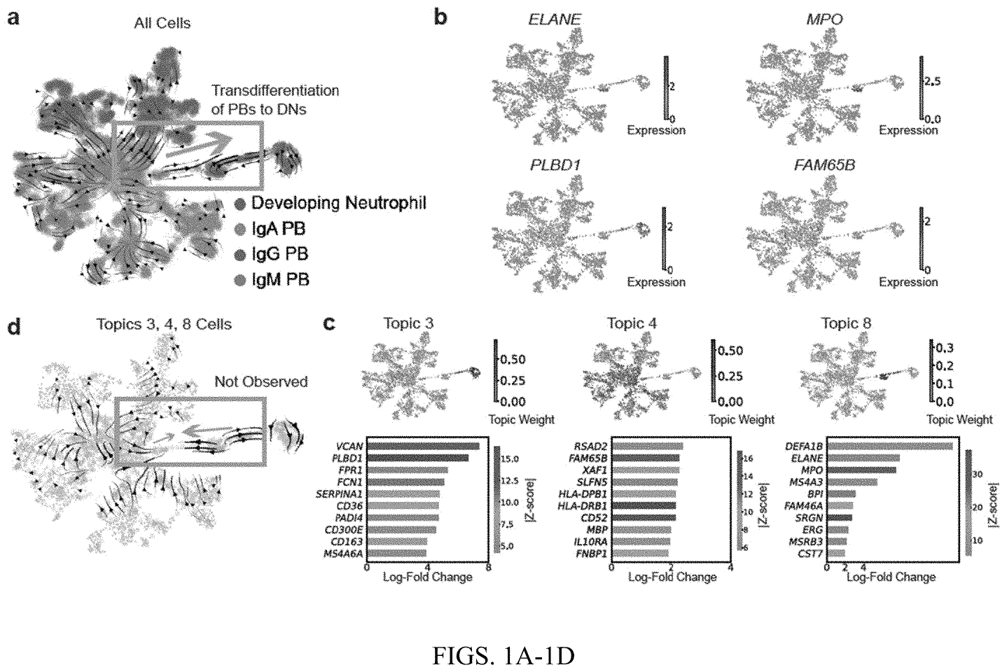

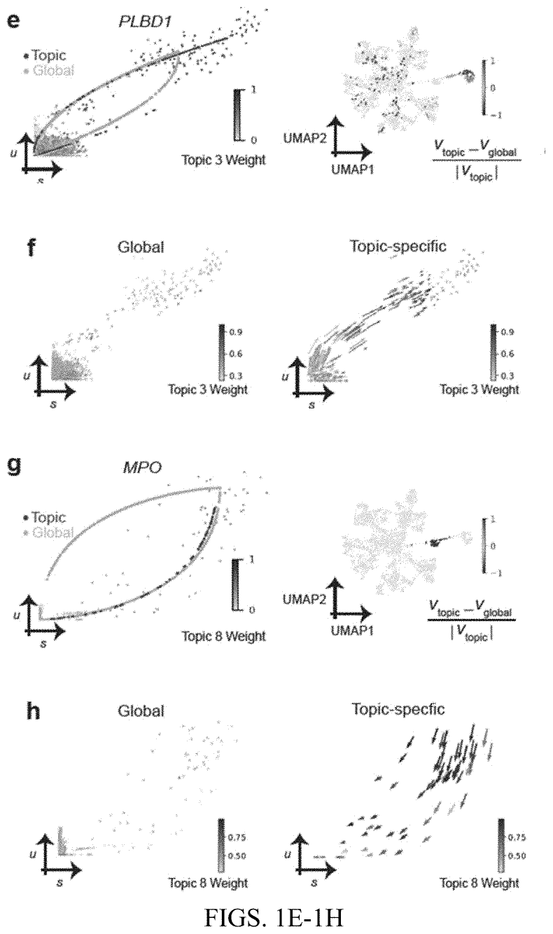

To explore this possibility, the inventors considered a highly cited scRNA-seq study of blood collected from COVID-19 patients [38], in which RNA velocity was used to infer a surprising, contested transdifferentiation of plasmablasts (PBs) into developing neutrophils (DNs) in the most severely ill patients [39, 40] (FIG. 1a). The inventors re-analyzed the relevant subset of the data and were able to reproduce the inferred PB-DN transition by following the recommended procedures for scVelo [5] (FIG. 1a, Supplementary FIG. 1a). Although cell-fate transitions are often characterized by genes whose expression is absent in a parent population, present in a terminal population, and increasing across the transition (or vice versa), the transitional genes highlighted in the study, such as MPO and ELANE, are not expressed in either the main plasmablast population or the seemingly terminal neutrophil population, but rather in a small population apparently between the two. The inventors also observed genes, such as PLBD1 and FAM65B, with inverse expression patterns (FIG. 1b).

To investigate how probabilistic topic modeling may affect the inferred RNA velocity dynamics in this context, the inventors fit the highly variable genes to an 11-topic model (FIG. 1c, Supplementary FIG. 1b-i). Using a published strategy [41], the inventors computed differentially expressed (DE), topic-specific genes associated with each topic (FIG. 1c, Supplementary FIG. 1b-i, Supplementary Table 1). Three of the topics, i.e., topics 3, 4, and 8, were high in cells associated with the PB-DN transition (FIG. 1c). Based on the published cell-type annotations, most cells with high topic-3 weights were DNs, consistent with the previously observed expression of topic-3 specific gene PLBD1 in neutrophils [42], though some cells with relatively high topic-3 weights were identified as IgM PBs. Cells with high topic-4 weights were primarily IgM PBs. Topic-8 high cells, characterized by expression of the neutrophil granule enzymes ELANE and MPO, were DNs in the apparently transitional population, close to IgM PBs. Using the dynamical model of scVelo, the inventors performed a focused RNA velocity analysis on the combined set of topic-associated cells and topic-specific genes for topics 3, 4, and 8. Unlike the global analysis, these results do not suggest a transdifferentiation from PBs to DNs (FIG. 1d).

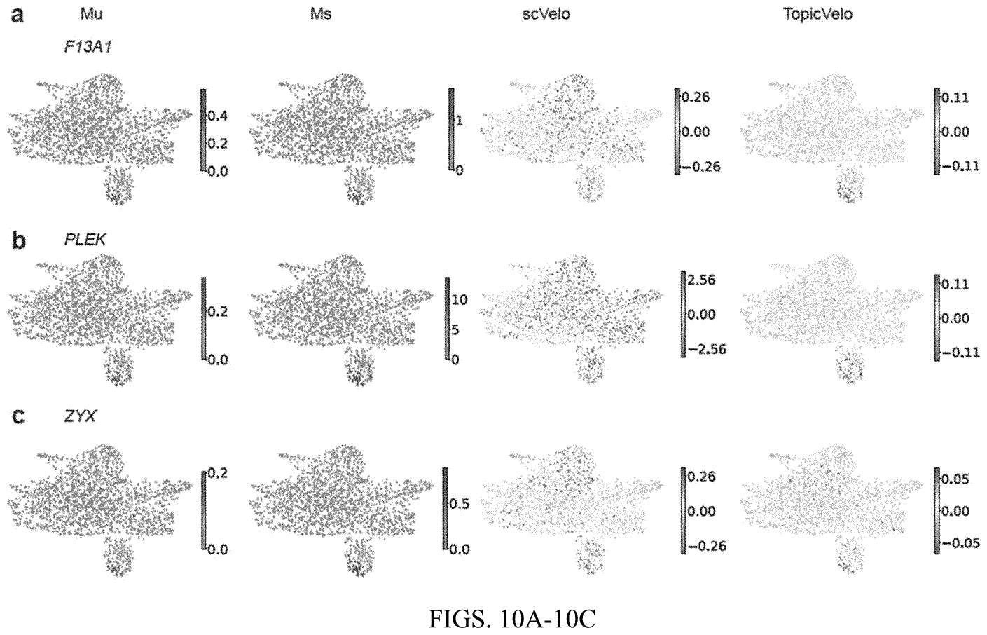

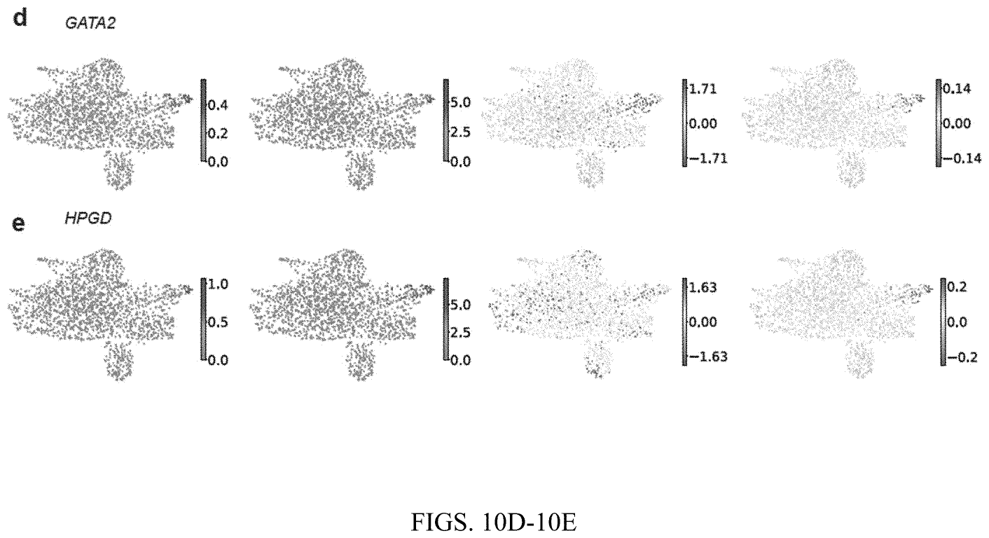

For each gene, the dynamical model of scVelo creates a phase portrait, i.e., it fits an ordinary differential equation to a scatter plot of cells representing their spliced versus unspliced transcript counts. While global phase portraits reflect overall trends, they do not capture topic-specific dynamics revealed in topic-specific phase portraits. For example, relative to the global phase portrait, the topic-3 specific phase portrait for PLBD1 captures a wider dynamic regime and predicts larger velocities for topic-3 associated cells (FIG. 1e), consistent with previously observed strong expression of PLBD1 in neutrophils [42]. Furthermore, relative to the globally inferred transition matrix, the topic-3 specific transition matrix shows a clearer up-regulation in cells with increasing topic-3 weights, which may correspond to the maturation of developing neutrophils (FIG. 10. In a different example, the phase plot of MPO shows little evidence of standard up-regulation, i.e., of cells with relatively high ratios of unspliced to spliced transcripts. (The pattern could also be an indication of “transcriptional boosting” [21], which is discussed later, but this is not a scenario handled by scVelo.) Yet, at the global scale, an up-regulation phase was inferred (FIG. 1g), though a visualization of the global transition matrix in the phase portrait for MPO reveals little signal that suggests transdifferentiation (FIG. 1h). In contrast, the topic-8 specific phase portrait indicates that MPO is down-regulated with decreasing topic-8 weight (FIG. 1g), which is also reflected in the topic-specific transitions (FIG. 1h) and the streamlines from the combined analysis (FIG. 1c). The analysis demonstrates that the choice of input cells and genes has significant effects on the inferred RNA velocities and cell-state transitions. Moreover, it suggests that topic modeling can help guide the selection of appropriate cells and genes for process-specific velocity inference and downstream analysis, thereby reducing the chance of erroneous discoveries.

### Example 2: Inferring and Integrating Process-Specific, Bursty Transcriptional Dynamics

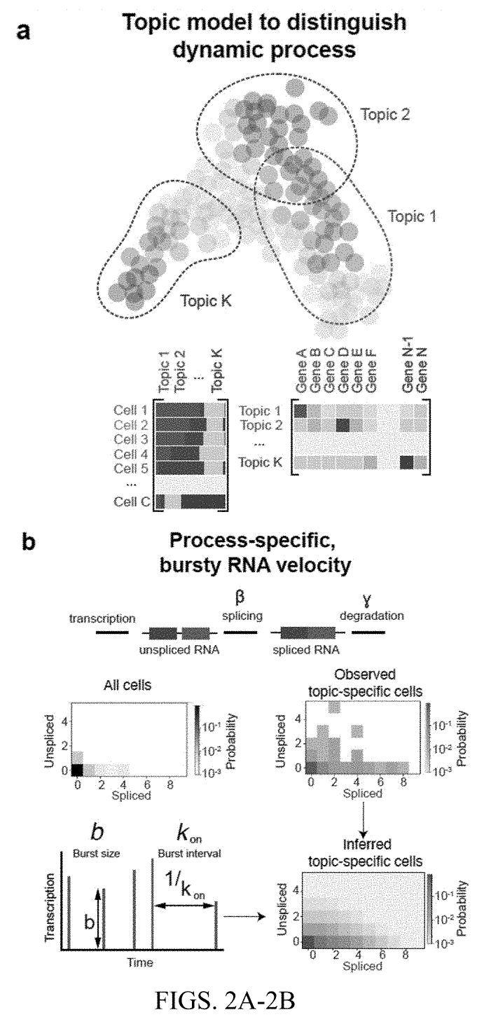

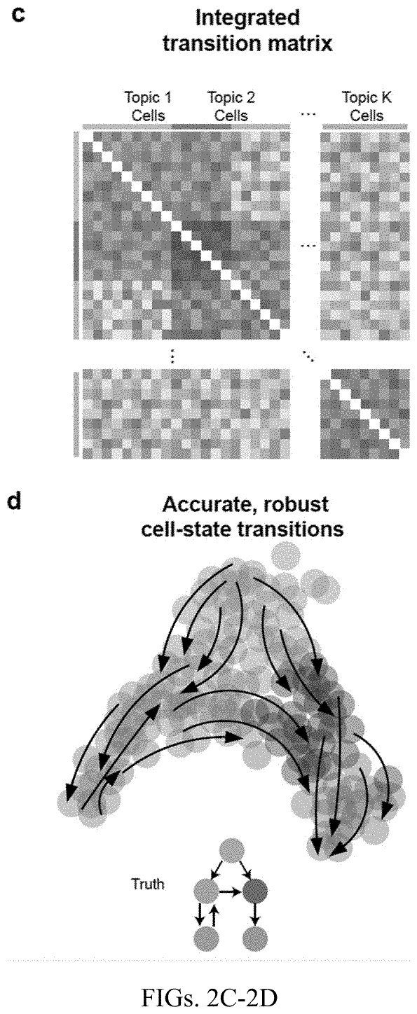

As the COVID data analysis and previous works illustrate, probabilistic topic modeling can effectively distinguish biologically relevant signals in scRNA-seq data [32-35]. Taking advantage of this capability, the inventors developed the RNA velocity method TopicVelo, which dissects simultaneous global dynamics into distinct, often overlapping, process-specific, dynamic regimes for parameter inference (FIG. 2a). In contrast to the ODE-based one-state model underlying scVelo, TopicVelo efficiently fits a more faithful physical model that accounts for transcriptional bursting (FIG. 2b) [43]. Finally, TopicVelo uses topic weights to integrate process-specific transition probabilities into larger-scale or global transition matrices (FIG. 2c). The result is much more robust, accurate inference and downstream analysis (FIG. 2d). In more detail, TopicVelo operates in the following three stages:

Process-specific inference. A topic model is fit to a counts matrix that includes, separately, spliced and unspliced transcripts. The result is a representation of each cell as a probability distribution over topics, while each topic is a probability distribution over individual genes (FIG. 2a). To emphasize how topic modeling is applied in this work, the inventors often use the term “process” to mean “topic” (or combination of topics). Process-associated cells, i.e., cells with relatively high weights in a topic, and process-specific genes, determined using previous strategies [32, 33, 35, 41], serve as input for inferring process-specific kinetic parameters. Within process-associated cells, process-specific genes can reveal a great deal of dynamic information that is hidden at the global scale and hence missed by existing methods (FIG. 2b).

The number of topics is a user-selected parameter, which, like clustering resolution, often has multiple, biologically meaningful settings. While metrics developed in other data science literature (e.g., [44-46]) offer insight into an appropriate range of topic numbers (Methods), they tend to suggest relatively high numbers of topics, which then encode redundant signals, possibly because the metrics were not designed for such noisy data. The inventors found that one coherence measure performed better when restricted to topic-specific genes (Methods). The inventors also found it helpful to use the literature to assess the interpretability of topic-specific gene programs. Regardless, in the applications, the inventors did not observe sensitivity of the overall results to the exact choice of topic number.

Bursty transcription model. The inventors developed a very efficient inference method to fit a model with geometrically distributed bursts of transcription, adapting a previous model for studying mRNA transport [36] (FIG. 2b). The chemical master equation of the model for a given gene is:

$$\frac{\partial p ( u , s , t )}{\partial t} = {k}_{on} [ {\sum }_{z = 0}^{u} {p}_{z} p ( u - z , s , t ) - p ( u , s , t ) ] \vert + β [ ( u + 1 ) p ( u + 1 , s - 1 , t ) - up ( u , s , t ) ] + γ [ ( s + 1 ) p ( u , s + 1 , t ) - sp ( u , s , t ) ] \qquad ( 1 )$$

where p (u, s, t) is the probability of observing a cell with u unspliced pre-mRNA and s spliced mature mRNA transcripts at time t; kₒₙ is the rate of the Poisson process governing the burst event; p_{z}, the probability of producing z unspliced pre-mRNA transcripts during a single burst event, is governed by a geometric distribution; β is the splicing rate; and γ is the rate of degradation of spliced mRNA. Parameters are initialized with the method of moments or another heuristic. For a given parameter setting, TopicVelo uses the efficient implementation of the Gillespie algorithm to estimate the full joint distributions of unspliced and spliced transcript counts. Then the Nelder-Mead algorithm is used to estimate the maximum likelihood parameter values and update the parameter settings (Supplementary FIG. 2, Methods).

Integration of process-specific dynamics. A key feature of TopicVelo is the capability to integrate process-specific transition matrices into a global transition matrix (FIG. 2c). First, from the inferred process-specific kinetic parameters, TopicVelo constructs process-specific transition matrices, based on previous approaches [4, 5]. Each of these transition matrices characterizes the probabilistic flow of process-specific transcriptional changes across process-associated cells. Then a larger scale or global transition matrix is constructed by linearly combining process-specific transition matrices, using the topic weights of cells. This strategy, which, in some aspects, is called late integration, enables locally important dynamics to be accurately recovered and then woven into larger-scale, complex trajectories. Selection of the topic weight threshold, which determines topic-associated cells, must balance an inherent trade-off between the benefit of separating dynamic processes and the risk of losing dynamic range. On one hand, using too low a threshold may have some of the drawbacks of global inference. On the other hand, using too high a threshold for determining topic-associated cells may distort parameter inference by reducing the dynamic range in the input. The threshold can be guided by the natural probabilistic interpretation of topic weights, as well as the commonly occurring bimodal distribution of cell weights for a given topic. TopicVelo also provides an intuitive heuristic based on the KL divergence between the distribution of a topic-specific gene over topic-associated cells and its distribution over all cells (Methods).

TopicVelo provides an alternative to late integration, which, in some aspects, is called early integration, which uses topic modeling only to focus inference on a relevant subset of genes created by combining topic-specific genes across all topics. Then all cells are used to infer kinetic parameters for these genes, and a unified, global transition matrix is computed via the standard approach. This strategy works effectively in systems with a simple, linear trajectory (which may also be modeled well by a small number of topics) because it ensures that, the full dynamic range of a gene across the trajectory is used for kinetic parameter inference. However, in more complex systems, parameter estimates can be distorted, as in standard RNA velocity inference, by irrelevant cells.

### Example 3: Inferring Complex Transitions in Human Hematopoiesis without Metabolic Labeling

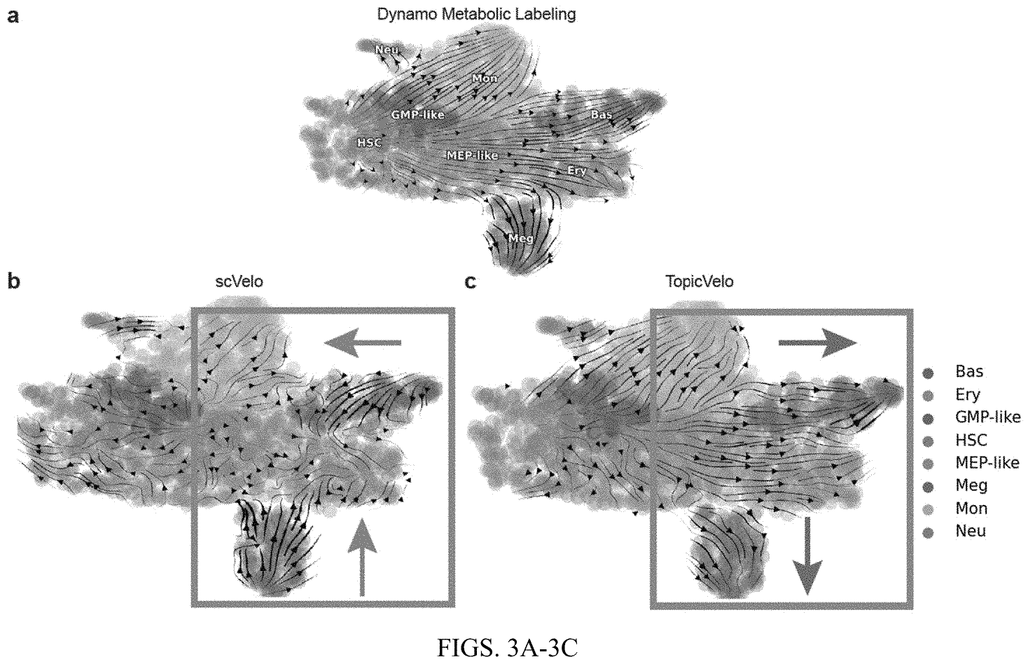

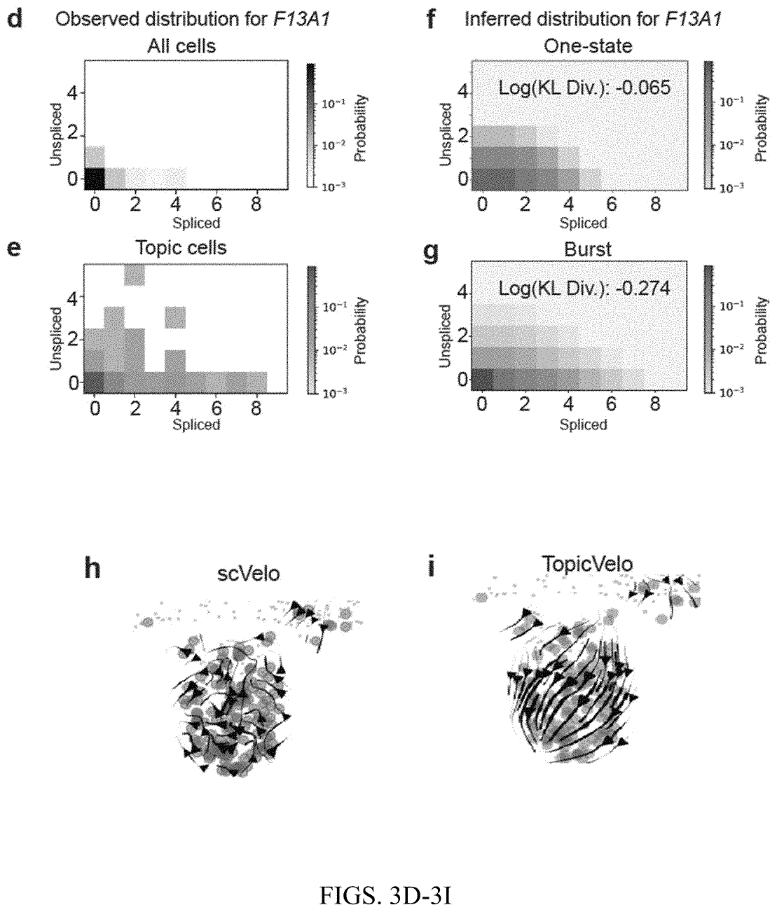

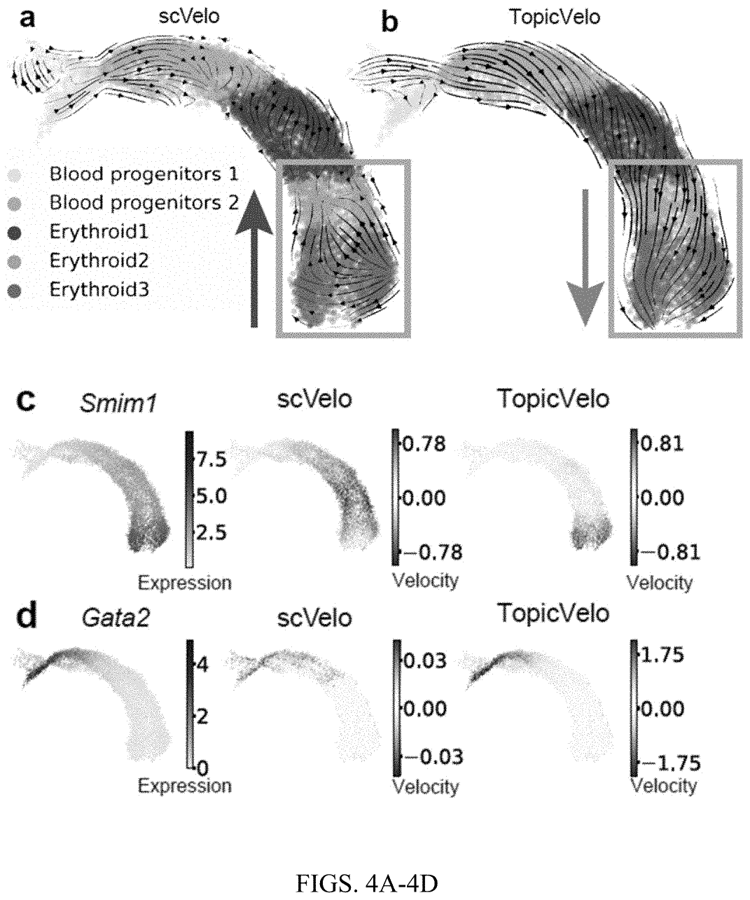

To test the effectiveness of TopicVelo, the inventors applied it to human hematopoiesis data from a recent study in which RNA velocity was extended to leverage single-cell metabolic labeling techniques that distinguish newly synthesized versus preexisting transcripts [20]. The published analysis reconstructed a complex, multifurcating trajectory of transitions (FIG. 3a), which scVelo fails to capture (FIG. 3b). Using TopicVelo on the data without the metabolic labels, the inventors were able to infer the correct transitions, including streamlines that accurately delineate the trajectories of monocytes, basophils, erythrocytes, and megakaryocytes (FIG. 3c).

The 8-topic model the inventors constructed from the data identifies gene programs associated with both known cell types (topics 1 and 3) and heterogeneous cell states during differentiation (Supplementary FIG. 3, Supplementary Table 1). In particular, topic 3 is correlated with megakaryocytes, which are known to synthesize plasma factor XIII [47], a subunit of which is coded for by the topic-3 specific gene F13A1 (Supplementary FIG. 3d, 4a). Though a global phase plot of F13A1 indicates little transcriptional activity (FIG. 3d), focusing on topic-3 associated cells brings the dynamical features of F13A1 into relief (FIG. 3e). Based on the assumption that the joint distributions of spliced and unspliced counts of megakaryocyte marker genes can be reasonably approximated as steady state, TopicVelo used the burst model to substantially improve upon the parameter estimates made by scVelo using the one-state model, including much more accurately recovering the experimental joint distribution of F13A1 over topic-3 associated cells (FIG. 3f, g).

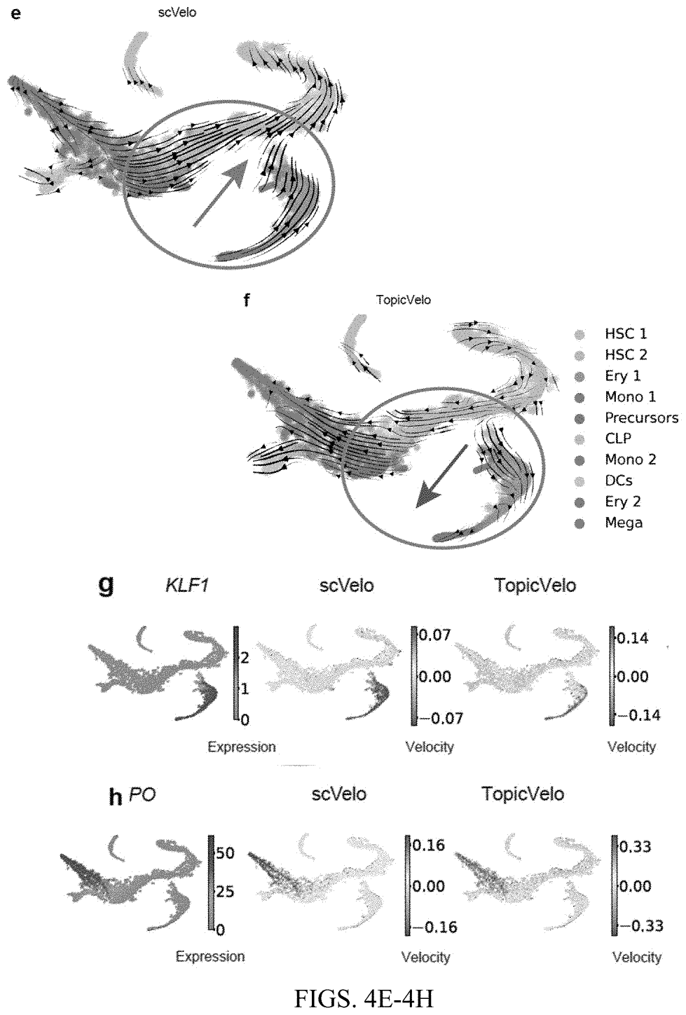

Moreover, for topic-3 specific genes, such as F13A1, PLEK, and ZYX, the positive velocities inferred by TopicVelo, but not the negative velocities inferred by scVelo, are consistent with exeri-mental evidence that these genes are up-regulated during megakaryocytic differentiation [48, 49] (Supplementary FIG. 4a-c). The result is that topic-3 specific streamlines estimated by TopicVelo are much more similar to the published streamlines in the same region (FIG. 3h, i). Similarly, for topic 1, which is strongly associated with basophils, TopicVelo predicts up-regulation of topic-specific genes GATA2 and HPGD during basophil development, whereas scVelo indicates down-regulation (Supplementary FIG. 4d, e). The TopicVelo estimates are consistent with previous experiments showing that GATA2 is critical for basophil development [50] and that HPGD is enriched in basophils [51]. These results demonstrate the capacity of TopicVelo to identify biologically meaningful dynamic genes and infer more biologically accurate velocities.

### Example 4: TopicVelo Handles Genes with Multiple Rate Kinetics and Recovers Erythropoiesis and Hematopoiesis Trajectories

Several studies have observed that some genes exhibit developmental stage- or time-dependent transcription rates, termed “multiple rate kinetics (MURK)” [4, 5, 21-23, 52]. In erythropoiesis during mouse gastrulation and in human bone marrow development, for example, certain genes exhibit a significant increase in the rate of unspliced mRNA production, or “transcriptional boosting” [21, 23, 52]. An analysis of previous experiments indicates that during mouse erythropoiesis, most MURK genes are up-regulated [21], yet RNA velocity inferred by scVelo from mouse erythropoiesis data suggests down-regulation of these genes, a major reason why scVelo erroneously produces reversed streamlines (FIG. 4a).

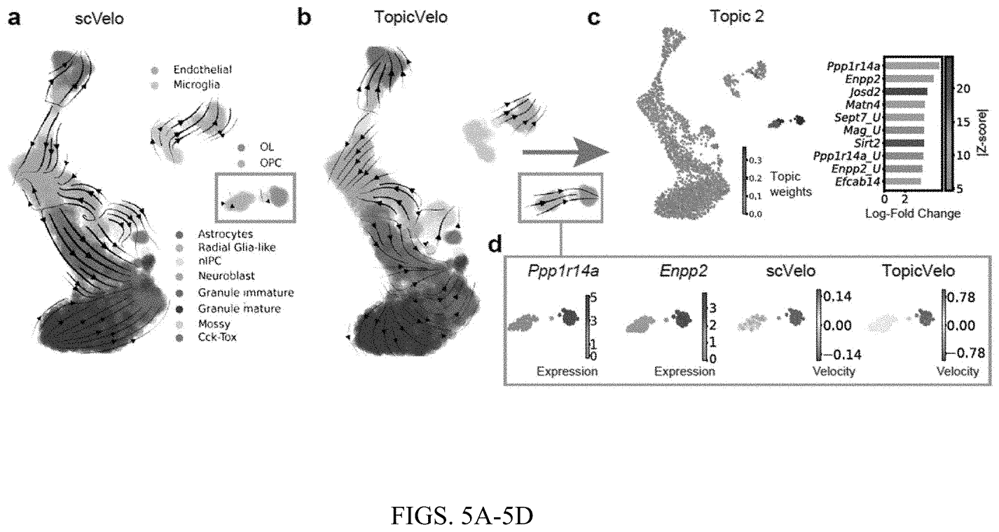

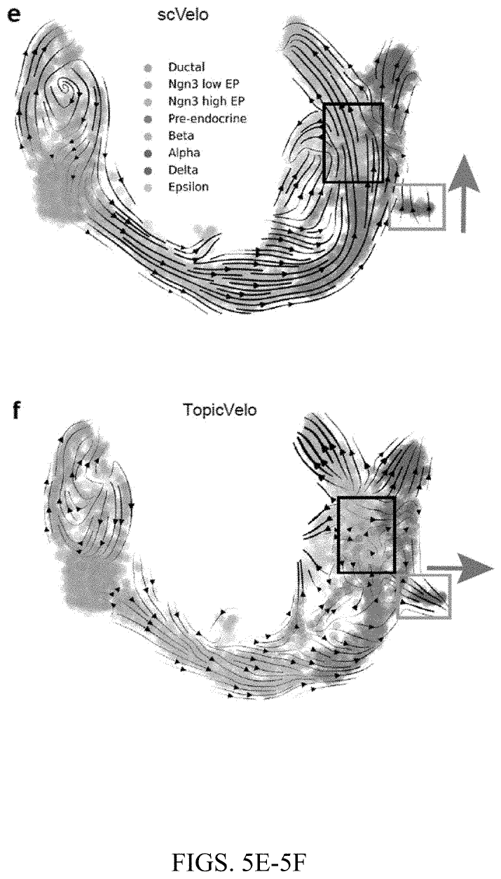

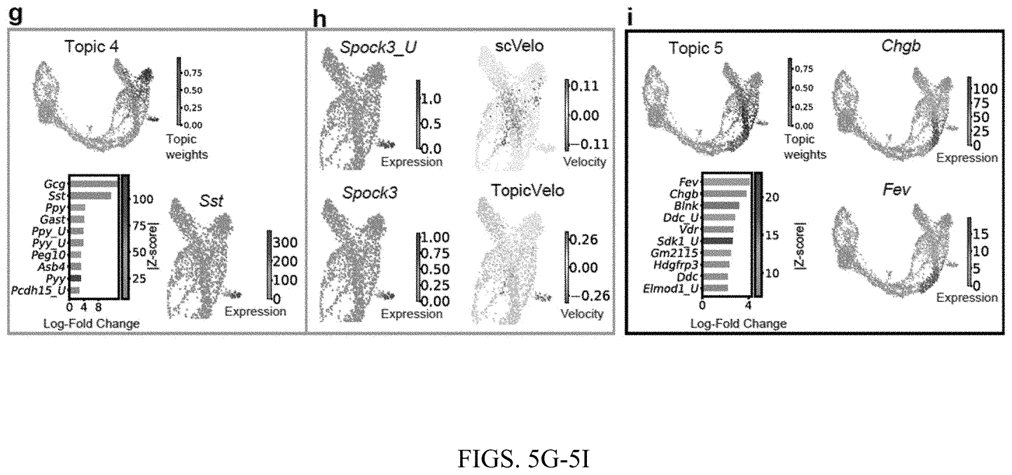

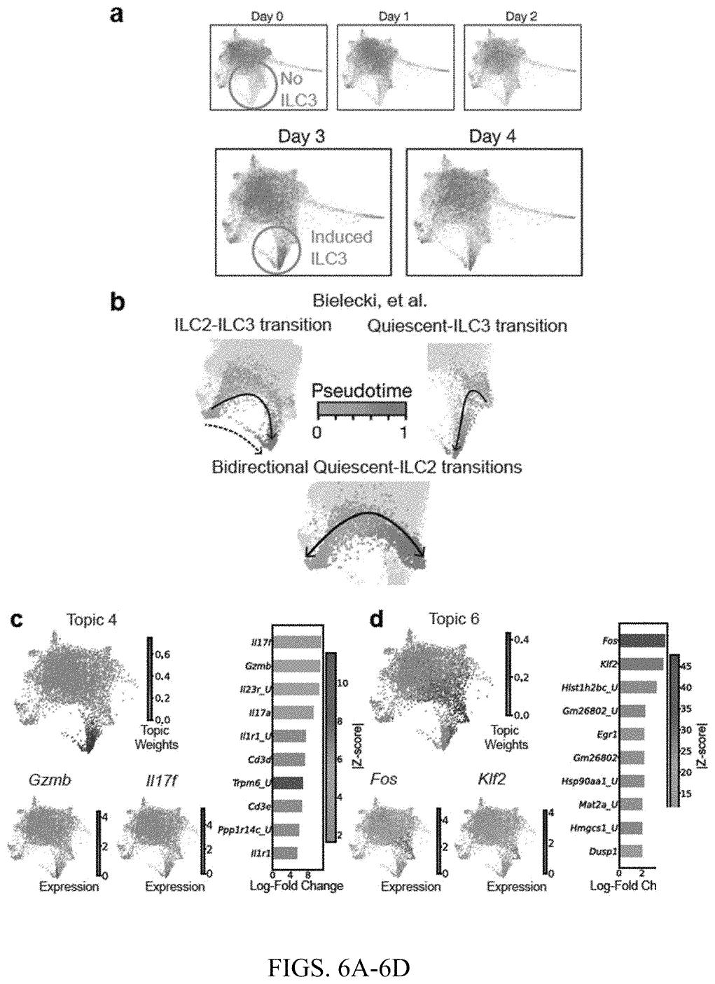

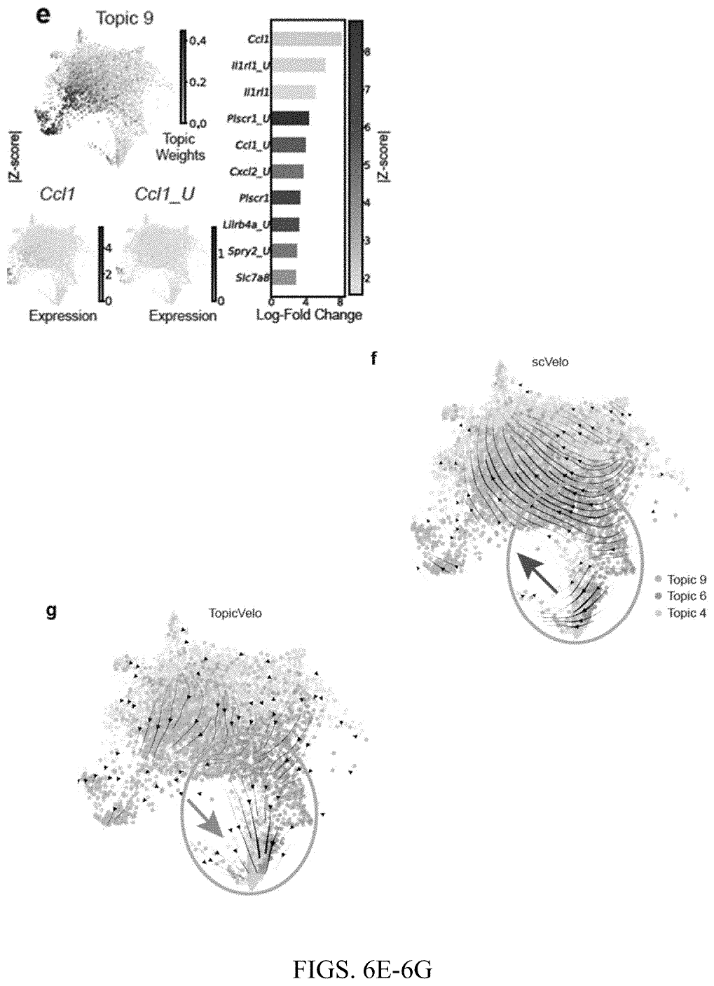

Using TopicVelo, the inventors analyzed the same mouse erythropoiesis data and obtained the expected trajectory (FIG. 4b). The inventors used only 2 topics since the cell types comprise only blood progenitors and erythroid cells (Supplementary FIG. 5, Supplementary Table 1). Topic 0 weights increase across the developmental process, while topic-0 specific genes are primarily up-regulated. Topic-0 features several MURK genes, including the archetypal red-blood cell genes Hba-x, Hbb-y [21], and their unspliced counterparts (Supplementary FIG. 5a). Though the coordinated changes and boosts in expression of MURK genes, such as Smim1, Dhrs11, Nxpe2, and Hemgn, may be due to time-dependent rate parameters that are not accounted for in either the TopicVelo or scVelo models, TopicVelo uniquely recovers biologically meaningful positive velocities for them (FIG. 4c, Supplementary FIG. 6a-c).

In opposition to topic 0, topic 1 weights decrease across the developmental process, along with the expression of topic-1 specific genes, such as Gata2, Fn1, Fscn1, Tead2, and Gsta4 (FIG. 4d, Supplementary FIG. 5b). These results corroborate previous observations that Gata2 is highly expressed in progenitors, with expression declining after erythroid commitment [53], and that Ccnd2 expression is anti-correlated with erythroid progression [54]. In contrast to the mixed, largely positive velocities inferred by scVelo for these down-regulated genes, TopicVelo infers strongly negative velocities (FIG. 4d, Supplementary FIG. 6d, e).

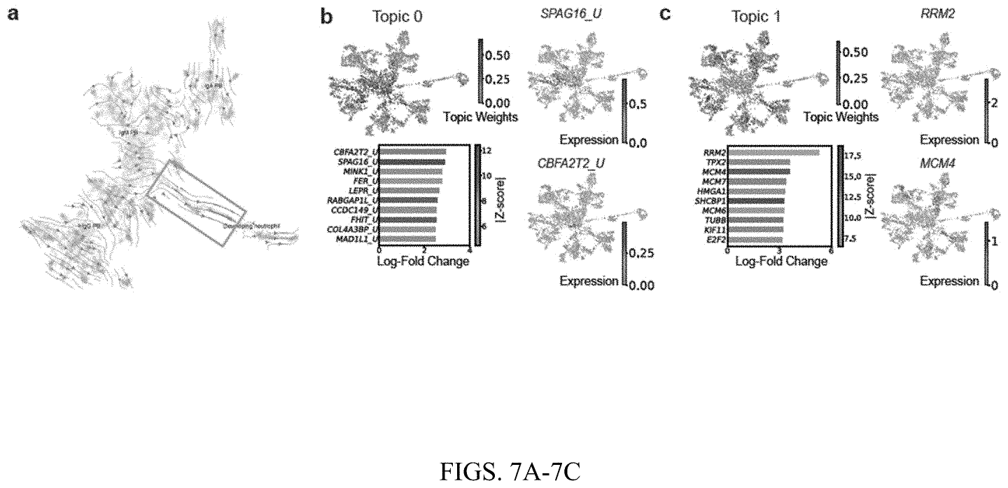

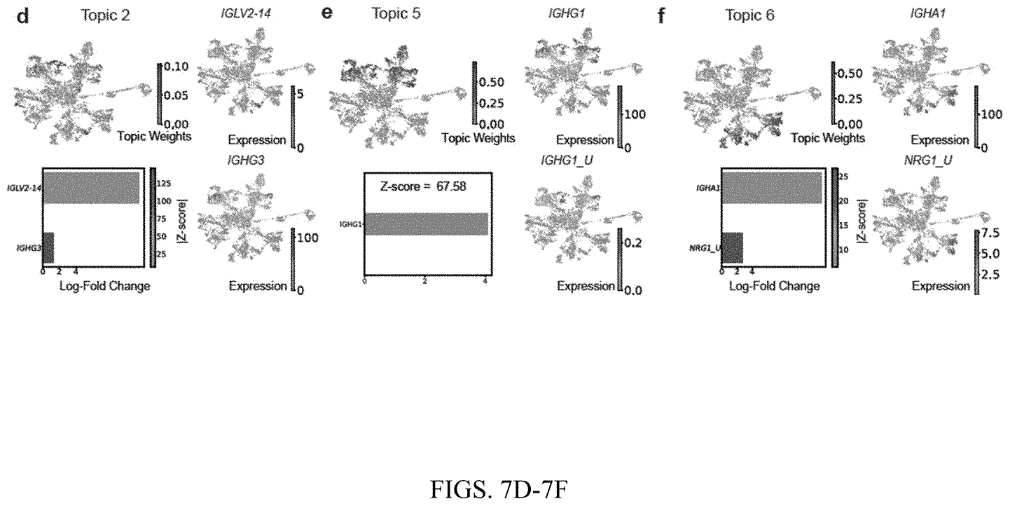

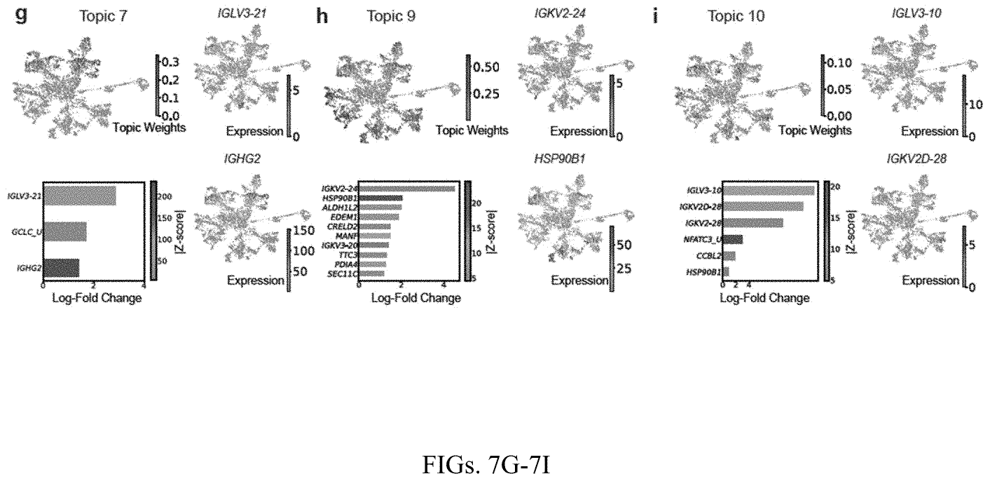

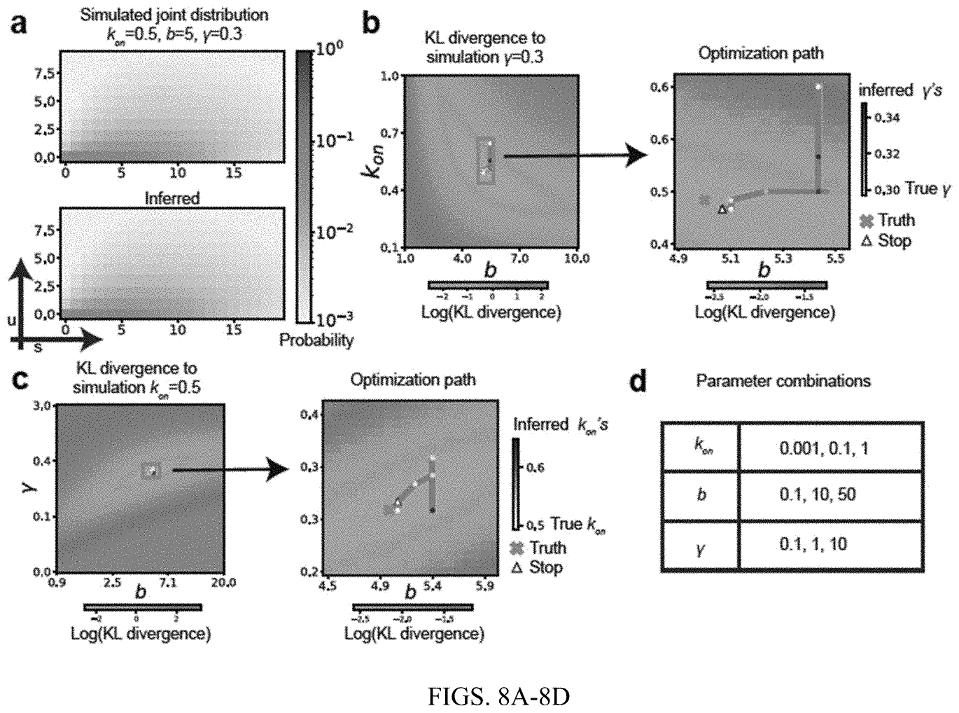

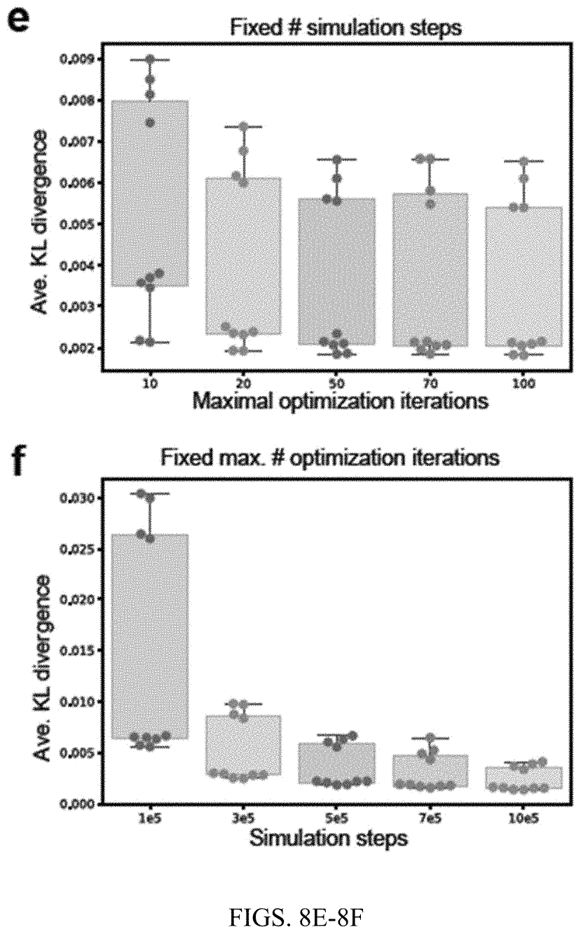

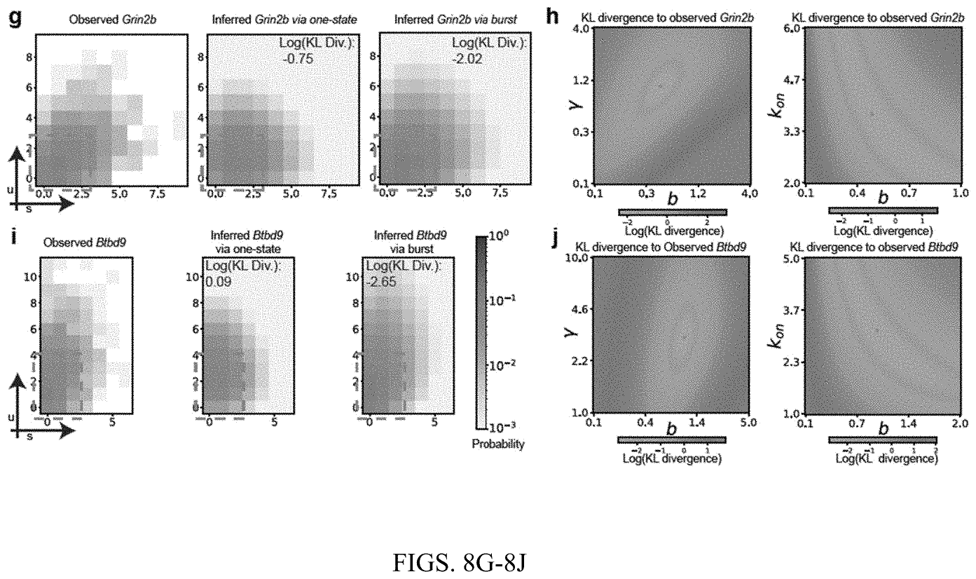

In the study of human bone marrow development, a careful pseudotime analysis (without RNA velocity) revealed a complex multi-furcating process of hematopoietic stem cell differentiation into terminal populations [52]. On this data, scVelo predict reversals of several established differentiation trajectories (FIG. 4e). In contrast, using 10 topics, TopicVelo recovers the expected trajectories and identifies key genes involved cell-fate commitments, without the prior knowledge of starting state required by pseudotime inference (FIG. 4f, Supplementary FIG. 7). The topics characterize different stages of development and terminal cell types. For example, topic 6 is relatively high in erythroid cells and includes the gene KLF1, which, as previously observed, is strongly correlated with ery-throid commitment [52]. Whereas scVelo predicts that early erythroid cells (Ery 1) down-regulate KLF1, TopicVelo accurately predicts that they up-regulate KLF1 (FIG. 4g). TopicVelo also captures the previously observed [52] up-regulation of MPO during monocyte commitment (FIG. 4h). Furthermore, as exemplified by CAI, a gene up-regulated in the peripheral blood erythroid cells [55]; IRF8, essential for monocyte development and function of dendritic cells [56]; SELP, a key marker gene for megakaryocyte development [57]; CRHBP, a marker for hematopoeitic stem cells that is down-regulated as during differentiation [58]; and AZU1, a chemotactic gene in monocytes [59]; TopicVelo highlights key developmental, topic-specific genes and predicts velocities for them that are more consistent with known biology (Supplementary FIG. 8, Supplementary Table 1).

### Example 5: TopicVelo Refines Lineage Predictions of Dentate Gyrus and Pancreas Development

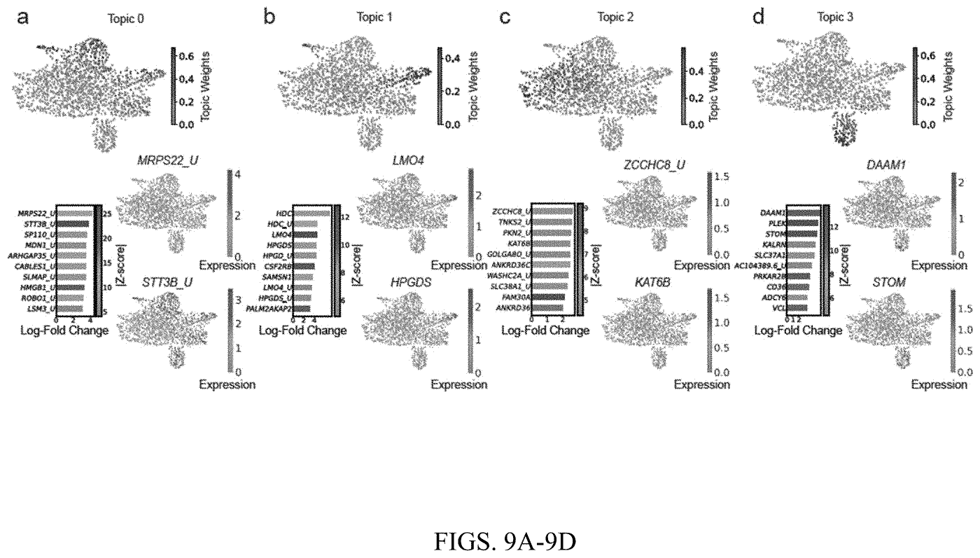

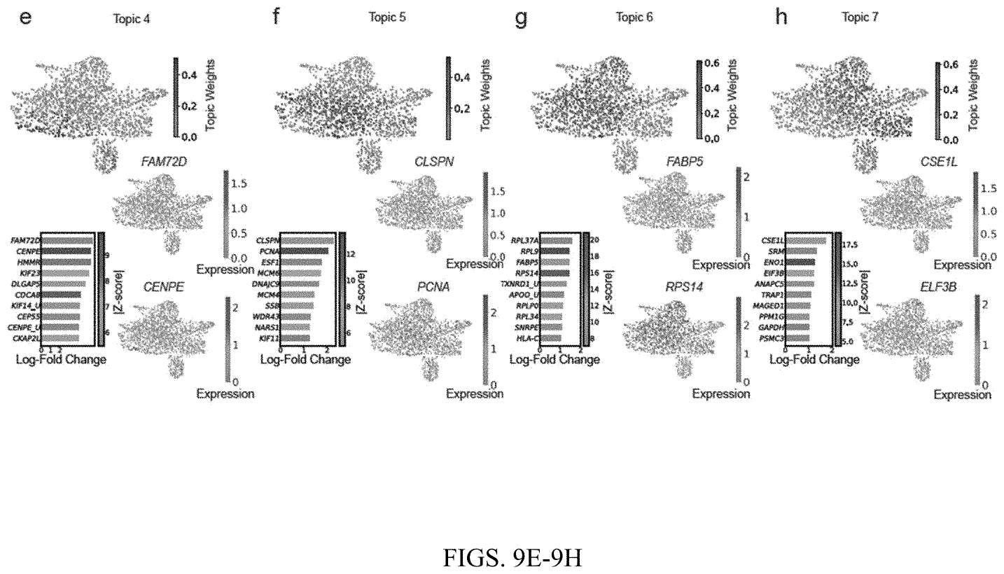

To test TopicVelo in settings previously handled well by scVelo, the inventors reproduced RNA velocity analyses of hippocampal dentate gyrus neurogenesis [5, 60] and pancreatic endocrinogenesis data [5, 61]. In the dentate gyrus data, scVelo recovers the main trajectory involving the astrocytes and granule lineages, though it fails to infer an established transition from oligodendrocyte precursor cells (OPCs) to oligodendrocytes (OLs) (FIG. 5a). With 10 topics, TopicVelo infers a similar main trajectory and also captures the transition in the smaller oligodendrocyte lineage clearly (FIG. 5b). Topic modeling results reveal a gene program (topic 2) associated with OPC and OL cells, and other programs corresponding to different stages of differentiation and terminal cell types (FIG. 5c, Supplementary FIG. 9, Supplementary Table 1). Topic-2 specific genes with high log-fold changes include Ppp1r114a, which was previously observed to be up-regulated during OL differentiation [62] and Enpp2, which has been shown to regulate oligodendrocyte differentiation in vivo in the hindbrain of developing zebrafish [63] (FIG. 5c, d, Supplementary Table 1). The observed expression in mouse also suggests an up-regulation of Enpp2 along the OPC-OL differentiation trajectory, which is more consistent with the positive velocities in OPCs inferred by TopicVelo than with the negative velocities inferred by scVelo in this population (FIG. 5d).

In the pancreatic endocrinogenesis data, scVelo reveals cell cycling in ductal progenitors and predicts their eventual commitments into α, β, and ϵ, but not δ, cells (FIG. 5e). Applying TopicVelo, the inventors obtained largely consistent results and also captured the differentiation of pre-endocrine cells into δ cells (FIG. 5f). The six topics in the model correspond to previously identified populations, including β, ϵ, Ngn3-expressing progenitor, and ductal cells (Supplementary FIG. 10). Topic 4 is strongly associated with both α and δ cells, and is characterized by genes, such as Gcg and Sst, which are well-known markers of α and δ cells, respectively [64] (FIG. 5g, Supplementary Table 1). Another topic-4 specific gene is Spock3, whose specific expression in human δ cells has been observed [65]. Though scVelo suggests very little up-regulation of Spock3 in the δ lineage, TopicVelo predicts specific, positive velocities in those cells (FIG. 5h). Pre-endocrine-associated topic 5 features specific expression of Chgb and Fev (FIG. 5i), genes previously reported to be enriched at the branch point of α and β commitments [66]. The co-existence of multipotent pre-endocrine cells with potential for both α and β cell fates could explain the noisy streamlines produced by TopicVelo in this regime. As a low-dimensional representation of the expectations of transitions, streamlines may not be able to visualize this type of complexity.

Overall, in these data sets, TopicVelo infers transitions that are largely consistent with those of scVelo. It also goes beyond those predictions, highlighting important developmental genes and accurately recovering key transitions in smaller cell populations.

### Example 6: TopicVelo Disentangles and Predicts Complex Immune Response Trajectories of Innate Lymphocytes

A previous computational and experimental study of a mouse model of psoriasis shows that pathological type-3 innate lymphoid cells (ILC3s) likely arise from other ILCs transitioning via multiple, convergent trajectories, including from ILC2s, a discovery that was confirmed with transgenic fate mapping [32]. The scRNA-seq analysis combines topic modeling with density-based pseudotime inference on data from multiple time points to disentangle transcriptional states among skin ILCs and model their trajectories during in vivo immune response. Cells were collected at day 0, before the initial subcutaneous administration of the cytokine IL-23, and then consecutively for 4 days, before each subsequent IL-23 injection. An ILC3 population that contributes to increasing skin lesions begins to emerge almost immediately and peaks at day 3 (FIG. 6a). The detailed analysis and experimental validations demonstrate multiple possible transitions to an ILC3-like state, including an ILC2-ILC3 transition that may occur via two routes and a quiescent-ILC3 transition, as well as a possible bidirectional quiescent-ILC2 transition (FIG. 6b).

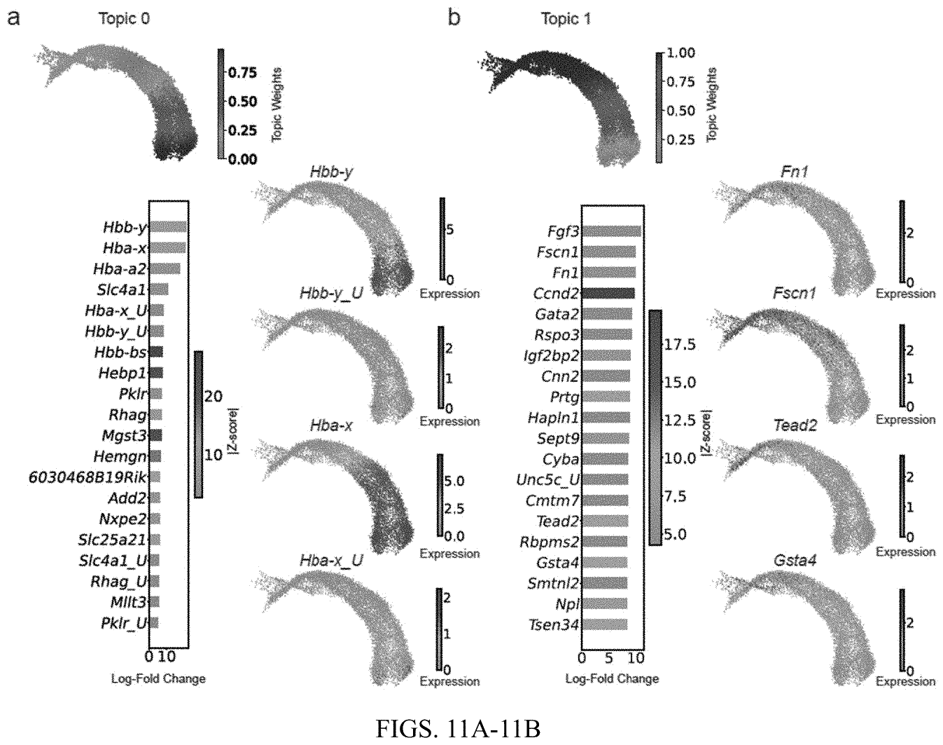

Focusing on the subset of ILCs from only day 3, the inventors assessed the capability of TopicVelo to predict these complex immune response trajectories without information from multiple time points or specification of root and terminal states. Topic modeling with 10 topics identifies three topics strongly associated with the ILC states involved in the transitions previously analyzed (FIG. 6c-e, as well as other topics characterizing this heterogeneous ILC landscape (Supplementary FIG. 11, Supplementary Table 1). Topic 4 is strongly associated with the ILC3-like cells, and characterized by proinflammatory, ILC3- and T_{H}17-specific genes, such as Gzmb, Il23r, and Il17a, and Il1r1 [67] (FIG. 6c). Topic 6 is enriched in cells and a gene program previously identified as “quiescent-like” [32], including Klf2, a transcription factor associated with T cell quiescence [68] (FIG. 6d). Topic 9 features ILC2- and T_{H}2-associated genes, such as Il1rl1 (ST2, the receptor for IL-33) [67], and chemokine genes, such as Ccl1, Cxcl2 and their unspliced counterparts (FIG. 6e).

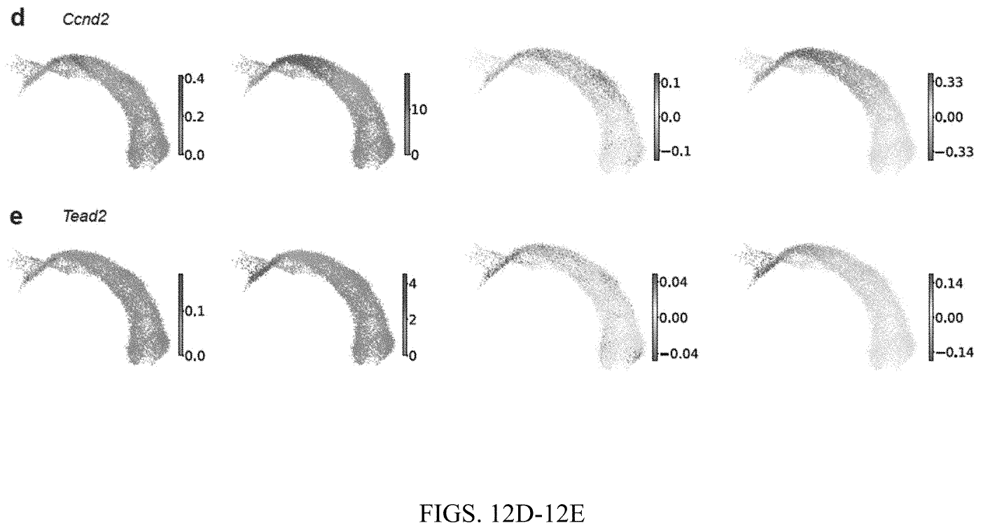

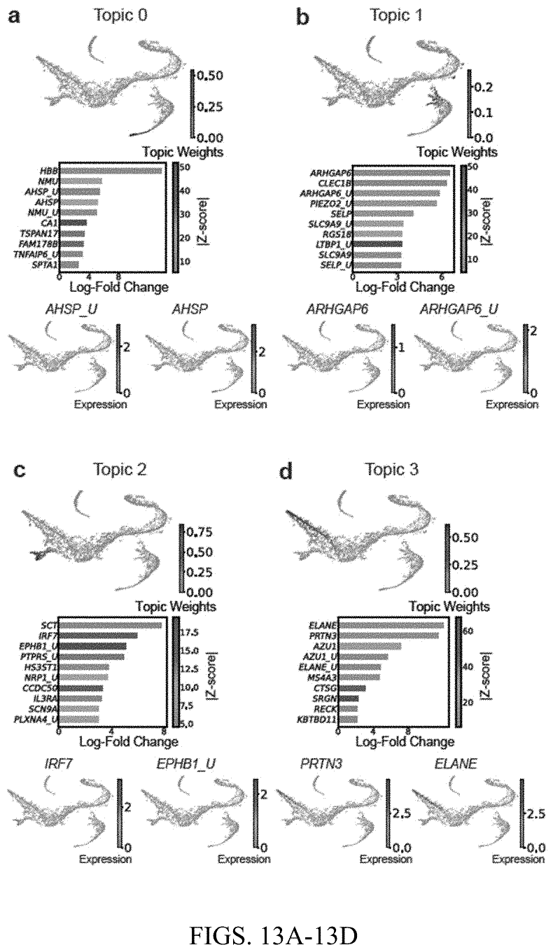

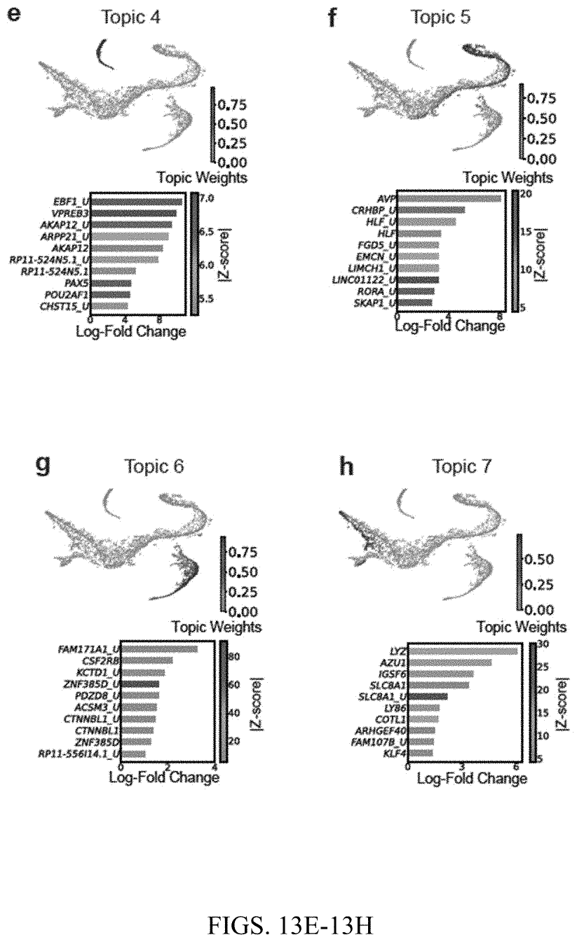

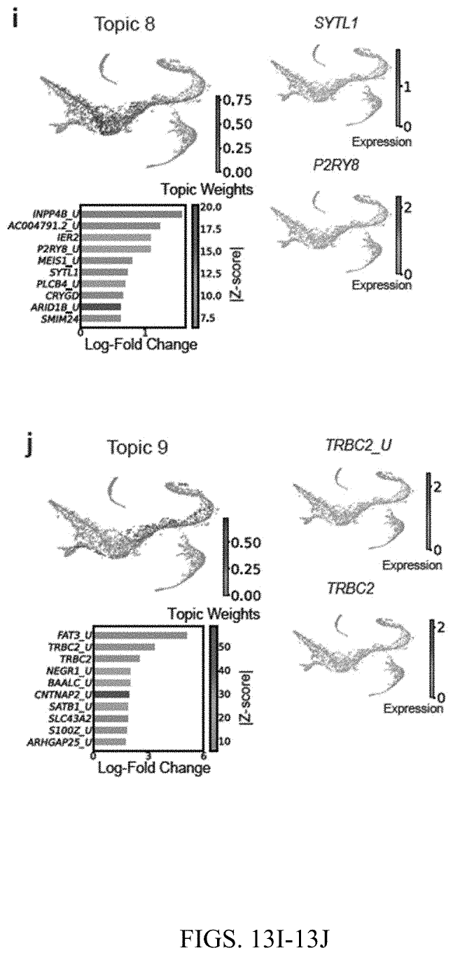

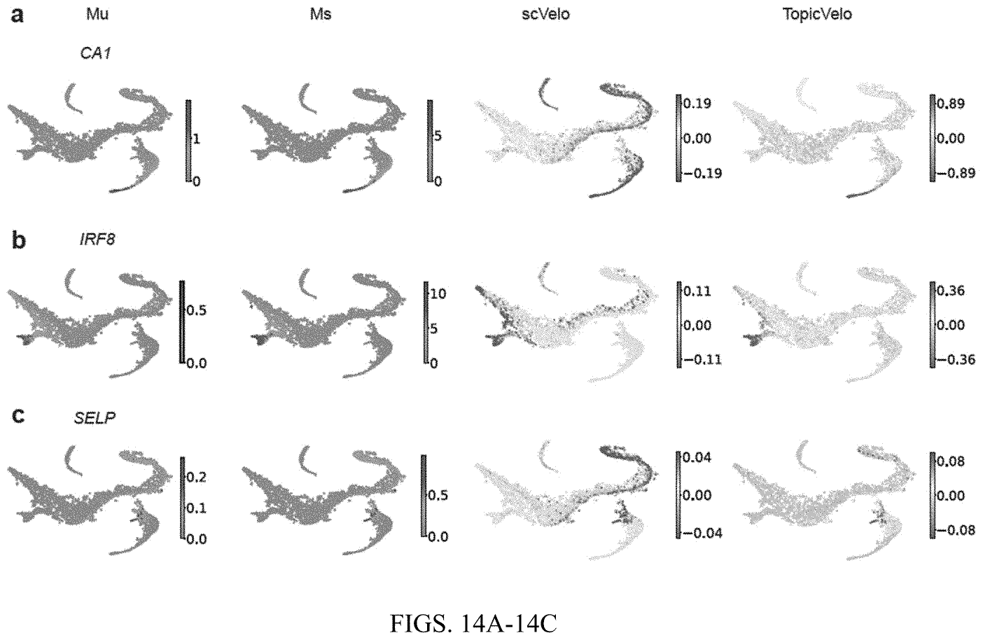

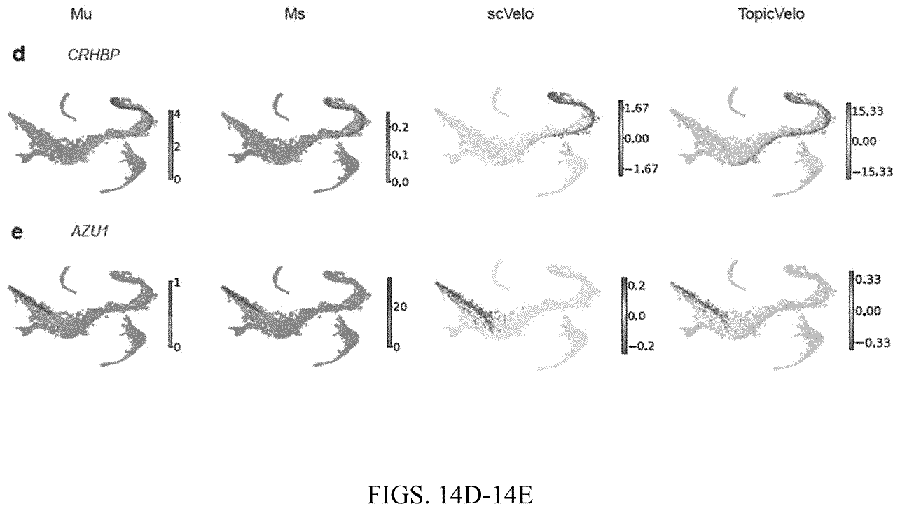

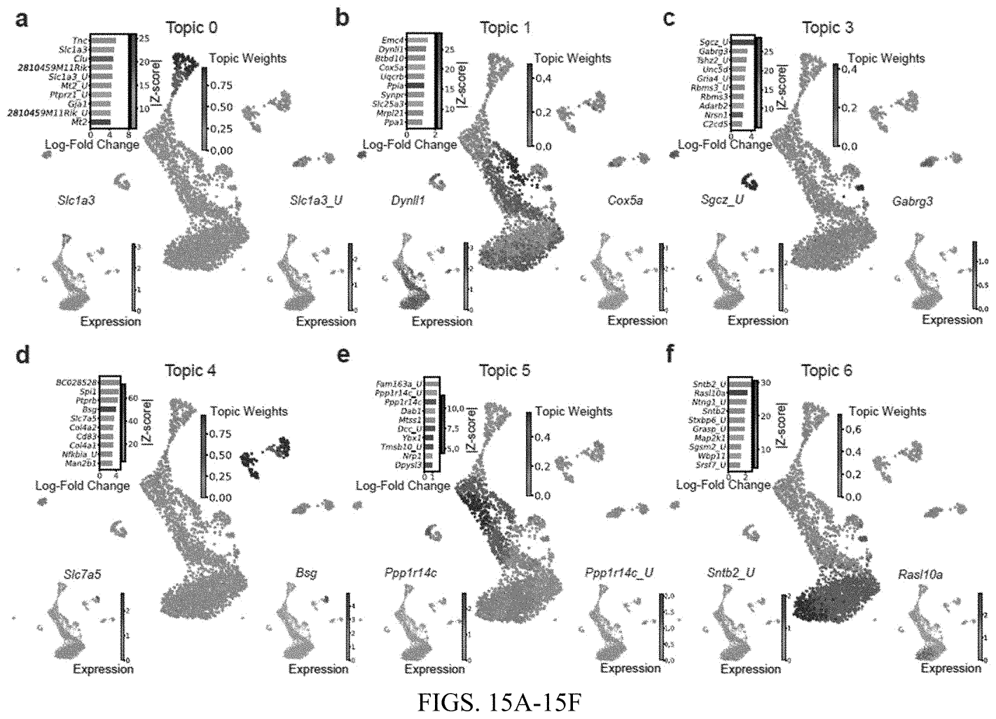

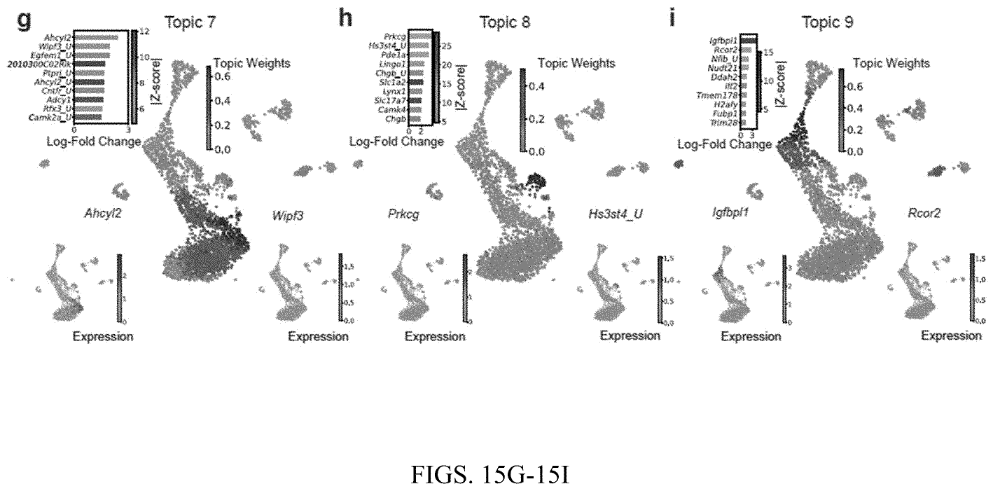

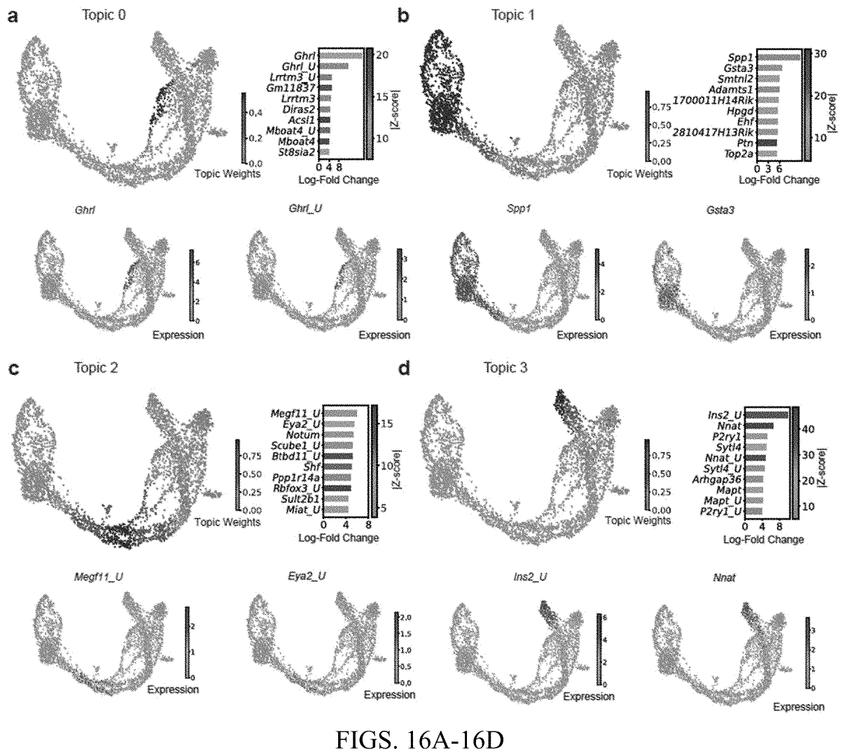

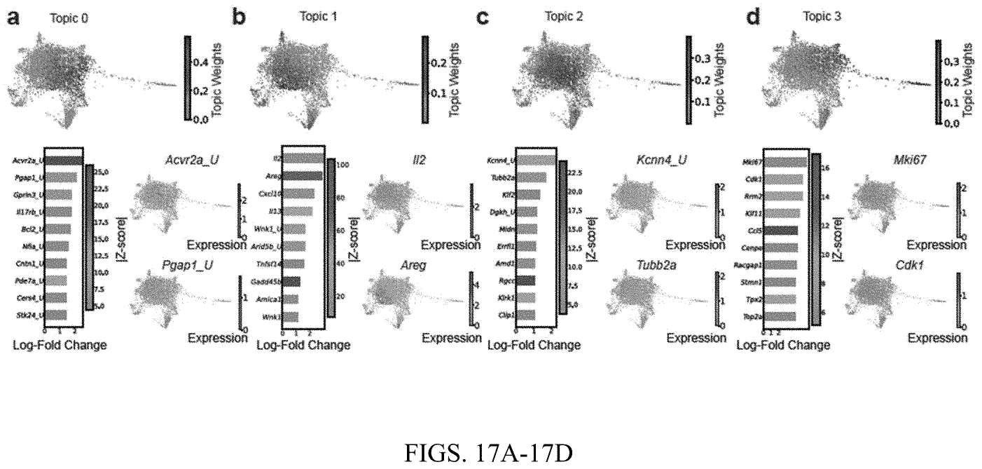

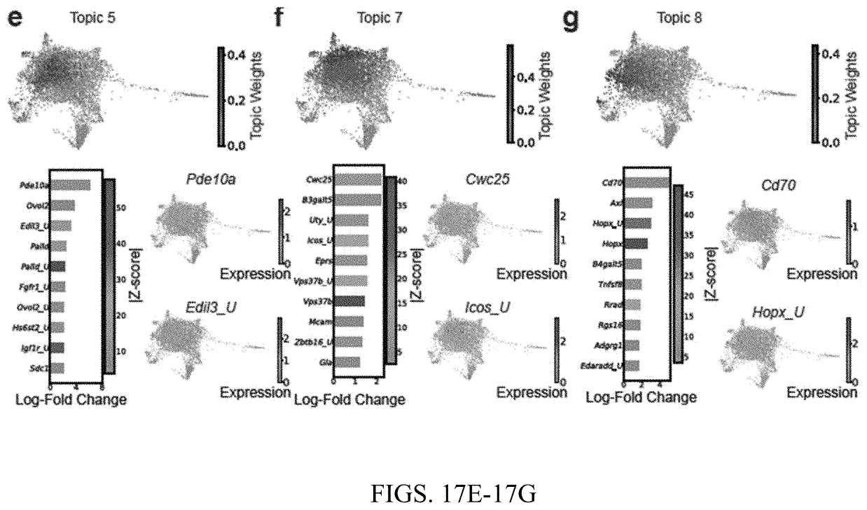

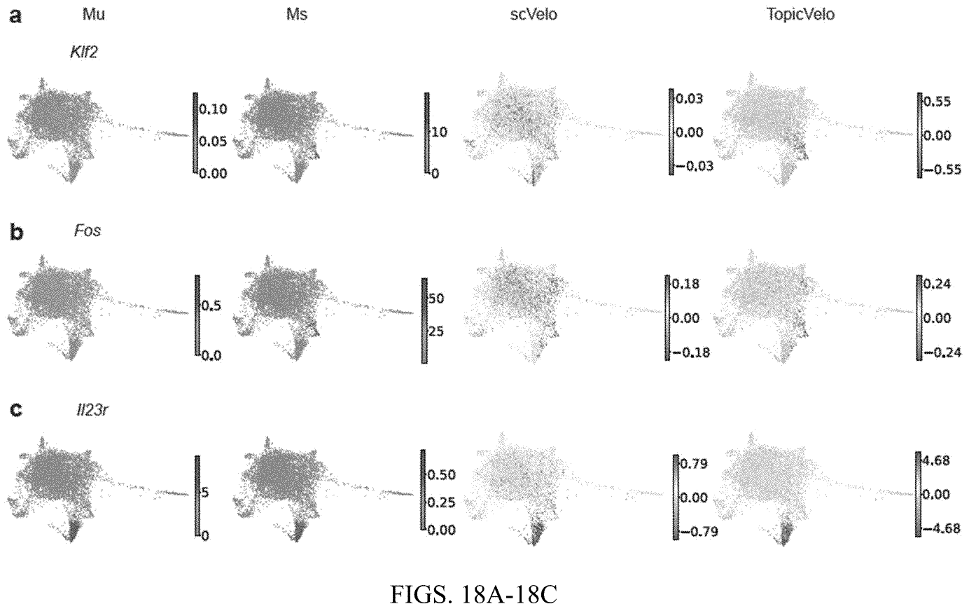

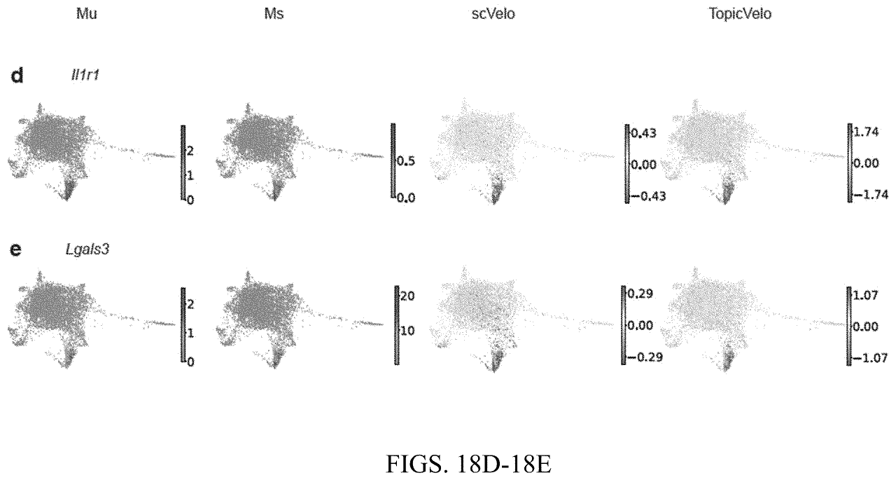

In an RNA velocity analysis focused on topics 4, 6, and 9, both TopicVelo and scVelo suggest a quiescent-ILC3 transition FIG. 6f, g) and predict the observed down-regulation of Klf2 and Fos [32] during the transition. However, only TopicVelo reveals the transition path of the ILC2-ILC3 trajectory or suggests a possible bi-directional quiescent-ILC2 transition (FIG. 6f, g). The discrepancies between TopicVelo and scVelo results are partially due to differences in velocity estimates. For example, the observed up-regulation of Il23r, Il1r1, and Lgals3 during ILC3 responses [32] are more faithfully captured by TopicVelo than scVelo (Supplementary FIG. 12a-12b).

These results suggest that the approach taken by TopicVelo may be more suitable for analyzing responding immune cells, which may be more likely than cells in developmental differentiation pro-cesses to exhibit unexpected functional plasticity and reflect varying contributions of simultaneous, very distinct dynamic processes.

### Example 7: TopicVelo Dissects and Integrates Bursty Transcriptional Dynamics for Complex Systems

Described herein, the inventors present TopicVelo, a new RNA velocity method that improves on the state of the art and conceptually complements other RNA velocity approaches. Existing methods typically include genes based on their fit to a velocity model [5, 15, 17], which makes strong assumptions about a globally determined steady state and can exclude genes that are informative specifically for locally dynamic processes. In contrast, by using topic modeling to discover biologically relevant gene programs or processes (“topics”) and the cells in which their activity levels are relatively high, TopicVelo hones in on genes that are informative for the kinetic parameters for different processes, while preventing cells that are not associated with a process from distorting its parameter estimates. To provide a global view of cell-state transitions, TopicVelo leverages the probabilistic topic weights to integrate process-specific transition matrices into a unified transition matrix.

TopicVelo infers gene-specific parameters of a transcriptional burst model by efficiently estimating the full joint distribution of unspliced and spliced gene counts given by a chemical master equation, thus explicitly accounting for higher-order moments. In contrast, the leading method scVelo [5] and others, which infer kinetic parameters based on ordinary differential equations (ODEs) from counts smoothed across cell neighborhoods in the k-NN graph, can distort second- or higher-order moments [24]. A recent method also incorporated a global burst model, fit via numerical gradient descent, rather than the simplex-based optimization in TopicVelo, though the study focused on analyzing the effects of gene-length dependent capture rates of unspliced RNA [37]. In the analyses of both simulated and real, biologically varied, single-cell datasets, the inventors find that the transcriptional burst model enables TopicVelo to more accurately estimate kinetic parameters, particularly for lowly expressed genes, which can play impactful biological roles [69, 70].

A critique [25] of the scVelo approach notes that smoothing actually occurs at multiple stages and leads to a potentially problematic, strong dependence of the parameters, especially in the dynamical model, on the structure of the k-NN graph, which ideally models the underlying manifold and is visualized in the UMAP embedding. At the gene level, TopicVelo circumvents this issue by inferring kinetic parameters from unsmoothed counts. Furthermore, by computing a different k-NN for each topic, TopicVelo loosens the coupling between the transition matrix and UMAP embedding. While TopicVelo, like scVelo, uses the inferred velocity matrix and a matrix of differences of smoothed spliced counts to compute transition probabilities, the TopicVelo framework also naturally permits (noisier) transition probabilities to be computed from differences of unsmoothed counts.

Using its dissection-then-integration approach, TopicVelo inferred robust, accurate dynamics in complex systems, including plastic immune responses and multi-furcating differentiation, without requiring multiple time points or the support of metabolic labeling. The combination of topic modeling with a stead-state transcriptional model may allow TopicVelo to implicitly handle some non-steady state contexts. The framework is also amenable to other topic models or transcriptional models that have been analyzed from a theoretical perspective (e.g., [71]). Moreover, because RNA velocity estimates are leveraged by recent, sophisticated pseudotime and dynamical inference methods (e.g., [Lange 2020, 20]), TopicVelo may improve their predictions by providing more accurate, biologically meaningful inputs for these approaches.

Future challenges include developing methods that merge the advantages of TopicVelo with other recent, complementary advances, such as the incorporation of a Bayesian framework for quantifying statistical uncertainty, which was developed for ODE velocity models [12], the improvement of transcript quantification to avoid biases [37, 72], and the incorporation of multi-omic data and models [9, 10].

### Example 8: Materials and Methods for Aspects Herein

Preprocessing scRNA-Seq Data

Genes were filtered with a threshold such that at least 20 cells have both spliced and unspliced mRNA transcripts. Based on the principal component analysis, Euclidean distances were computed on the top 30 PCs and a K nearest-neighbor graph was constructed (30 nearest neighbors by default) on logarithmized spliced mRNA counts. Then the top 2000 highly variable genes were selected. The count matrices were size-normalized using the total counts and the first and second moments of each cell were estimated over the k-NN graph. These procedures were performed via scVelo:

scVelo.pp.filter_and_normalize(adata, min_shared_counts=20)scVelo. pp.moments (adata, n pcs=30,n neghibors=30)

### Topic Modeling and Differential Expression Analysis Via Topic Modeling

When applying the multinomial topic model to scRNA-seq data, each cell xi is drawn from a multinomial distribution

xᵢ₁, . . . ,x_{iM}|tᵢ˜Multinom(tᵢ;π_{i,1}, . . . ,π_{i,M})∀1≤i≤C (2)

where C is the number of cells, M is the number of genes, xim is number of mRNA transcripts for the mth gene in the ith cell and tᵢ=Σₘ₌₁^{M}xᵢₘ is total number of transcripts in cell i. The multinomial probabilities are

$${π}_{i , m} = {\sum }_{k = 1}^{K} {L}_{ik} {F}_{mk} \qquad ( 3 )$$

where K is the number of user-specified topics (or gene programs), L∈R^{C×K} is the cell topic weight matrix, Lᵢₖ is the proportion that topic k contributes to the counts in cell i; F∈R^{m×K} is the gene program matrix and Fₘₖ is the probability of gene m occurring in topic k.

For a given K, by exploiting the equivalence of maximum likelihood estimation between the Poisson non-negative matrix factorization (NMF) and the multinomial topic model [41], a suitable loss function is the negative of the log-likelihood of the Poisson NMF after discarding the terms that are not related to L and F [29],

$$- log {p}_{NMF} ( {x}_{im} \vert L , F ) = - log ( \frac{{( {L}_{i}^{T} {F}_{m} )}^{{x}_{im}} {e}^{- {L}_{i}^{T} {F}_{m}}}{{x}_{im} \text{?}} ) \qquad ( 4 )$$

$$minimize l ( L , F ) = {\sum }_{i = 1}^{c} {\sum }_{m = 1}^{M} {L}_{i}^{T} {F}_{m} - {x}_{im} log ( {L}_{i}^{T} {F}_{m} ) \\ subject to L \geq 0 , F \geq 0 \qquad ( 5 )$$

$$\text{?} \text{indicates text missing or illegible when filed}$$

where Lᵢ and Fₘ are the column vectors (of size K) containing the i^{th} row of L and the m^{th} row of F. In other words, the optimal L and F are fitted such that accounting for heterogeneity of every cell over the topics and contributions of individual genes for each topic, the input count matrix should be recovered on expectation.

Since the count is generated by a Poisson model, the differentially expressed genes can be identified by computing the log fold changes (LFC) for each gene in topic k, defined as

$${k}^{′} = {argmin}_{{k}^{′} \neq k} \vert \frac{{F}_{mk}}{{F}_{{mk}^{′}}} - 1 \vert \\ LFC ( k ) = {log}_{2} ( \frac{{F}_{mk}}{{F}_{{mk}^{′}}} ) \qquad ( 6 )$$

the posterior distribution of the LFC and local false sign rates[73] are then estimated with MCMC and stabilized with adaptive shrinkage. These procedures were performed using FastTopics:

topic_modelfit<-fittopic_model(count_matrix,k=K)

de_results<-deanalysis(topic_model_fit,count_matrix)

For each dataset, the inventors constructed a count matrix by stacking the raw spliced count matrix and the raw unspliced count matrix of the top 2000 highly variable genes. A topic model was fit with a specified topic number K. The optimal number of topics was chosen by the computing statistics of established measures [44-46] using tomotopy [74]. The first metric the inventors considered uses average distance among topics to measure stability:

$$corre ( k , {k}^{′} ) = \frac{{\sum }_{m = 1}^{M} {F}_{mk} {F}_{{mk}^{′}}}{\sqrt{{\sum }_{m = 1}^{M} {( {F}_{mk} )}^{2}} \sqrt{{\sum }_{m = 1}^{M} {( {F}_{{mk}^{′}} )}^{2}}} \\ ave\_cosine \_dis = \frac{{\sum }_{k = 1}^{K} {\sum }_{{k}^{′} = k + 1}^{K} corre ( k , {k}^{′} )}{K ( K - 1 ) / 2} \qquad ( 7 )$$

where corre(k, k′) is the standard cosine distance between topics k and k′. A smaller ave cosine dis indicates more stability. The second metric the inventors considered is information divergence between all pairs of topic pairs:

$$D ( k ‖ {k}^{′} ) = \frac{1}{2} {\sum }_{m = 1}^{M} {F}_{mk} log ( \frac{{F}_{mk}}{{F}_{{mk}^{′}}} ) + {F}_{{mk}^{′}} log ( \frac{{F}_{{mk}^{′}}}{{F}_{mk}} ) \\ ave\_info \_dis = {argmax}_{K} \frac{{\sum }_{k , {k}^{′}} D ( k ‖ {k}^{′} )}{K ( K - 1 )} \qquad ( 8 )$$

where D(k∥k′) is the Jensen-Shannon distance between two topics. A bigger ave info dis indicates more independence and the topic modeling contains more information overall. The inventors also tested a few coherence measures which are based on the pointwise mutual information (PMI) of top genes [46]:

$$PMI ( {g}_{m} , {g}_{{m}^{′}} ) = log \frac{P ( {g}_{m} , {g}_{{m}^{′}} ) + ϵ}{P ( {g}_{m} ) \cdot P ( {g}_{{m}^{′}} )} \qquad ( 9 )$$

where P(gₘ, g_{m′}) is probability of observing both genes gₘ and g_{m′} in a cell. P(gₘ) and P(g_{m′}) are the probabilities of observing gene gₘ and g_{m′} in a cell respectively. ϵ is a small number (e.g. 10⁻¹²) to prevent the PMI from reaching 0. If the top N genes are considered, the UCI coherence is calculated by:

$${C}_{UCI} = \frac{2}{N ( N - 1 )} {\sum }_{m = 1}^{N - 1} {\sum }_{{m}^{′} = i + 1}^{M} PMI ( {g}_{m} , {g}_{{m}^{′}} ) \qquad ( 10 )$$

A smaller difference between C_{UCI} and 0 indicates better topic coherence and higher probability that the top genes are co-expressed. To prevent overfitting, the inventors also consider the Akaike information criterion (AIC) and the Bayesian information criterion (BIC):

AIC=2·M·(K−1)−2· [symbol] _{K} (11)

BIC=(K−1)·M·log(C)−2· [symbol] _{K} (12)

where L_{K} is the log-likelihood of model when the number of topics is K.

In addition, interpretability, i.e. a reasonable number of potentially biologically meaningful differentially expressed genes is another important criterion. For most of datasets, topic genes were selected from the differentially expressed genes if the lfsr is below 0.001 and the LFC is either above 0.5 or below −0.5. If either the spliced or the unspliced or both satisfies the criterion, the gene is considered as a topic gene. This criterion is a very conservative estimate and in practice produces 50-150 topic genes for each topic.

### RNA Velocity Parameter Estimation Via the One-State Model

The one-state transcription model is governed by the following master equation

$$\frac{\partial p ( u , s , t )}{\partial t} = α [ p ( u - 1 , s , t ) - p ( u , s , t ) ] + β [ ( u + 1 ) p ( u + 1 , s - 1 , t ) - up ( u , s , t ) ] + γ [ ( s + 1 ) p ( u , s + 1 , t ) - sp ( u , s , t ) ] \qquad ( 13 )$$

in which α is the rate of transcription, the steady-state distribution when β≠γ [75] is the product of two independent Poisson distributions for u and s respectively:

$$p ( u , s ) = \frac{{( \frac{α}{β} )}^{u} {( \frac{α}{γ} )}^{s}}{u \text{?} s \text{?}} exp ( - \frac{α}{β} - \frac{α}{γ} ) \qquad ( 14 )$$

$$\text{?} \text{indicates text missing or illegible when filed}$$

Then the log likelihood for observing C cells at steady state with unspliced and spliced counts {uᵢ, sᵢ}ᵢ₌₁^{C} governed by a set of kinetic parameters is:

$$\begin{matrix}\vert ℒ ( {\{ {u}_{i} , {s}_{i} \}}_{i = 1}^{C} \vert α , β , γ ) = ln {\prod }_{i = 1}^{C} p ( {u}_{i} , {c}_{i} ) \\ = - C ( \frac{α}{β} ) + ln ( \frac{α}{β} ) {\sum }_{i = 1}^{C} {u}_{i} - {\sum }_{i = 1}^{C} ln ( {u}_{i} \text{?} ) - \\ C ( \frac{α}{γ} ) + ln ( \frac{α}{γ} ) {\sum }_{i = 1}^{C} {s}_{i} - {\sum }_{i = 1}^{C} ln ( {s}_{i} \text{?} )\end{matrix} \qquad ( 15 )$$

$$\text{?} \text{indicates text missing or illegible when filed}$$

The maximum likelihood estimate of γ/β is:

$$0 = \frac{\partial }{\partial ( α / γ )} ℒ ( {\{ {u}_{i} , {s}_{i} \}}_{i = 1}^{C} \vert α , β , γ ) \\ \frac{α}{γ} = \frac{1}{C} {\sum }_{i = 1}^{C} {s}_{i} = 〈 s 〉 \\ similarly , \\ \frac{α}{β} = 〈 u 〉 \\ \to \frac{γ}{β} = {γ}^{′} = \frac{〈 u 〉}{〈 s 〉} \qquad ( 16 )$$

where [symbol] ⋅ [symbol] denotes expectation. [symbol] s [symbol] and [symbol] u [symbol] are the average abundance of u and s over all cells in steady-state from data. scVelo uses a more cell-focused approach in the implementation of the stochastic mode, the moments for each cell are computed from a nearest neighbors graph. For each gene, an generalized least squares is performed by solving a system of linear equations involving the first and second moments for the cells in steady state (top right corner of the (u, s)-plot) [5]. This does not agree with the underlying biophysical model exactly though deviation is small in practice for unsmoothed data since the first moment is invariant over time under the steady-state assumptions. However, different choices for constructing the k-NN graph can affect the parameter estimate in unexpected ways [24, 25].

### RNA Velocity Parameter Estimation Via the Geometric Burst Model

To estimate the steady-state joint distributions, the inventors implemented a Gillespie algorithm to simulate the master equation [76] in Python, accelerated via Numba [77]. For a trajectory with burn in period t_{burn-in} and total simulation time tₜₒₜₐₗ, the probability of observing a cell with u unspliced mRNA and s spliced mRNA for a given gene in the steady state p (u, s) is

$$p ( u , s ) = \frac{1}{{t}_{total} - {t}_{burn - in}} {\int }_{{t}_{burn - in}}^{{t}_{total}} δ ( u , s , t ) dt \qquad ( 17 )$$

where δ(u, s, t)=1 if the cell has u unspliced count and s spliced count at time t, 0 otherwise.

To infer the kinetic parameters governing the dynamics, the inventors initialize the parameters with the method of moments which have been previously derived [36, 37]

$$\hat{b} = \frac{〈 {u}^{2} 〉}{〈 u 〉} - 1 \qquad ( 18 )$$

$${\hat{k}}_{on} = \frac{〈 u 〉}{b} \qquad ( 19 )$$

$$\hat{γ} = \frac{〈 u 〉}{〈 s 〉} \qquad ( 20 )$$

in which the moments are estimated from the observed distribution. Then the KL divergence was minimized using the Nelder-Mead algorithm implemented in SciPy [78] to find the optimal kinetic parameters. In some cases, the method of moment estimate is a local minimum that is close to the global minimum and the optimizer can get stuck. In this case, \hat{k}ₒₙ/3, 3·\hat{b}, and \hat{γ} may be used as starting point to search for the global minimum. Convergence criterion was chosen such that once the relative change in KL divergence between subsequent iterations was smaller than 1/1000 or when a maximum number of iterations has passed.

To verify the correctness of this estimation approach, the inventors computed the joint distribution at kₒₙ=0.5, b=5, γ=3 and compare it with the joint distribution with the inferred parameters; the two distributions are nearly identical (Supplementary FIG. 2a). To visualize the path of the optimization, the inventors computed the KL divergence landscape in kₒₙ v.s. b with γ fixed at 0.3 and the KL divergence landscape in γ v.s. b with kₒₙ fixed at 0.5 and visualize the path of the optimizations; the inventors observed the optimizer ended very close to the ground truth (Supplementary FIG. 2b, c).

The inventors aimed to find choices of simulation steps and maximal iterations that perform well for this inference scheme. The inventors considered a total of 27 parameter combinations across different dynamical regimes in which average mRNA abundances are over the span of several orders of magnitudes (Supplementary FIG. 2d). First, the inventors fixed the number of simulation steps at 5×10⁵ and analyzed how different maximal optimization iterations affect the performance of the inference. For each maximal iteration, the inventors simulated 10 replicas of the 27 combinations and computed the average KL divergence within each replica (Supplementary FIG. 2e). The inventors observed that the performance stopped improving when more than 50 maximal iterations were used. Similarly, the inventors fixed the number of maximal iterations at 50 and examined how different simulation steps affected inference. The inventors observed the performance improvement becomes marginal when more than 5×10⁵ steps were used (Supplementary FIG. 20. Therefore, the inventors chose a combination of 5×10⁵ reactions and a maximal iteration of 50 to be used for parameter inference of scRNA-seq data. In both settings, the bimodality of the average KL divergence might be due to the optimizers being stuck at local minima. To ameliorate this effect in real datasets, the inventors perform 5 independent optimizations for each gene and uses the set of parameters that corresponds to the lowest KL-divergence for downstream analysis.

When applied to scRNA-seq data, the joint distribution of spliced and unspliced counts were tallied from the raw data or the size-normalized data rounded to the nearest integer. A time-invariant splicing rate β=1 has been assumed for all genes under the steady-state assumption; kₒₙ, b and γ were estimated for each gene. To illustrate the validity of the parameter inference scheme, the inventors took the observed distributions of Grin2b from the granule mature cells in the dentategyrus dataset [60] which the inventors used as a proxy for steady state since these cells are terminally differentiated. The inventors noticed the geometric burst model recovers a joint distribution that is closer to the observation than the one-state model by capturing the diffusiveness of the distribution more accurately (Supplementary FIG. 2g). The inferred parameters from the burst model for Grin2b are located within a regime of low KL divergence as shown on the KL divergence landscapes (Supplementary FIG. 2h). The inventors performed the same analysis for Btbd9 and the observation was similar (Supplementary FIG. 2i, j).

### Integration of Process-Specific Dynamics

Late integration: For each topic within a given dataset, topic cells are defined as cells above a certain topic weight. The inventors used the following procedures to identify a reasonable range for the choice of a topic threshold. For a given topic k, the inventors denote the set of cells above the n^{th}-percentile as A_{n,k}⁺, and the set of cells below or equal the n^{th}-percentile as A_{n,k}⁻. Note that A_{n,k}⁺∪A_{n,k}⁻=A, where A is the set of all cells. The inventors compute the KL divergence from A_{n,k}⁺ to A and from A_{n,k}⁻ to A for integers n from 1 to 99 for the topic genes with the highest log-fold changes. For the KL divergence from the distribution over A_{n,k}⁺ to the distribution over A as n decreases, the KL divergence approaches 0. A sharp decline in the KL divergence is observed for a relatively large n=nk. Regime above this n corresponds to a regime for which the full dynamic range is not properly accounted for. For the KL divergence from joint distribution over A_{n,k}⁻ to the distribution over A, as n increases, the KL divergence approaches 0. A sharp decline in the KL divergence is observed for a relatively small n=n⁻. Regime below this n corresponds to a regime in which the inventors risk including cells not meaningfully associated with the gene programs. The interval [nk, nk] is a natural and simple heuristic as the range of suitable thresholds for topic k. For majority of the topics, the inventors observed a range around [30, 70] in which both KL divergence are relatively flat. In some cases, this range was observed be around [75, 95] and these topics often correspond to a rare cell type.

Kinetic parameters of topic genes are inferred on topic cells within the topic-specific steady state. In scVelo's implementation, the upregulation and downregulation steady-states for a given gene are the top right corner and the bottom left corner of the phase plot respectively. The exact determination is dependent on arbitrary expression thresholds; the default setting uses the [5,95] percentile. Instead of assuming each gene has its own set of steady-state cells, TopicVelo uses cell topic weights for a gene program as a criterion for choosing steady state cells which are more robust and biologically meaningful because the genes in the gene programs have similar expression patterns. Furthermore, the inventors observed the downstream analysis was not drastically impacted by this choice as long as the topic cells are selected properly. Therefore, the inventors invoke the steady-state assumption for all topic cells for simplicity. Then the velocity vector for i in topic k can be computed as v_{i,k}=(v_{i1,k}, v_{i2,k} . . . , v_{iM,k}), v_{im,k}=ũᵢₘ−γ_{m,k}\tilde{s}ᵢₘ where v_{im,k} is the topic-specific velocity for cell i and gene m, y′_{m,k} is the topic-specific degradation rate for gene m. ũᵢₘ and \tilde{s}ᵢₘ are the unspliced and spliced counts for cell i and gene m respectively.

Then a cosine similarities matrix between the velocity vectors and differences in the spliced expression is computed:

\tilde{p}_{ij,k}=cos(s_{j,k}−s_{i,k},v_{i,k}) (21)

where s_{j,k} is a vector of spliced expression of cell j for topic k genes. While it is more natural and preferable to use unsmoothed counts to compute velocity and to compute cosine similarity when the data is less affected by technical noises, smoothed counts were used for streamline visualization to reduce the noises. The first moments of the smoothed data is not as distorted as the higher-order moments thus the resulting velocity can be reasonably interpreted as an effective velocity. Furthermore, the inventors did not observe significant differences in the overall trends between the visualizations. Then an exponential kernel is applied to the cosine similarities \tilde{p}_{i j,k}:

$${p}_{ij , k} = \frac{1}{{z}_{ik}} exp ( \frac{{\tilde{p}}_{ij , k}}{{σ}^{2}} ) \\ where {z}_{i k} = {\sum }_{j} exp ( \frac{{\tilde{p}}_{ij , k}}{{σ}^{2}} ) \qquad ( 22 )$$

is the normalization factor and the kernel width parameter is σ over a topic-specific k-NN graph computed using the global PCA. p_{i j,k} is the topic-specific transition probability between cell i and cell j for topic k. Let p_{i j,k} be the i j-th entry of the topic-specific transition matrix Tₖ. Then the global transition matrix T is obtained through a linear combination of topic-specific transition matrices weighted by the cell's renormalized topic weights:

$${\tilde{L}}_{ik} = \frac{{L}_{ik}}{{\sum }_{{k}^{′} ϵ \{ {i}_{k} \}} {L}_{{ik}^{′}}} if k ∈ \{ {i}_{k} \} , 0 otherwise \qquad ( 23 )$$

$$T = {\sum }_{k = 1}^{K} {T}_{k} diag ( {\tilde{L}}_{k}^{T} ) \qquad ( 24 )$$

$$diag ( {\tilde{L}}_{k}^{T} ) = {l}_{C} \cdot ( {l}_{C}^{T} ⊗ {\tilde{L}}_{k}^{T} ) \qquad ( 25 )$$

where {iₖ} is the set of topics cell i was included in during late integration. diag(\tilde{L}ₖ^{T}) is the diagonal matrix where the i^{th} entry is the topic weight of cell i in topic k re-normalized over {iₖ}.

Early integration: Topic genes for all topics are aggregated to be considered dynamical genes. Then velocity parameters of these dynamical genes are inferred using all cells. The velocities are then used to construct a global transition matrix as done in scVelo.

### Analysis of the Human COVID-19 PBMC Data

After preprocessing of the scRNA-seq data, the inventors applied scVelo's dynamical model to generate the streamlines and obtain the gene-specific global phase portraits. The inventors then applied topic modeling with 11 topics and identified topic genes. Then the inventors assigned each cell a topic for which it has the highest weight in. For topics 3 and 8, the inventors computed topic-specific phase portraits for topic genes and topic-specific transition matrices for topic cells with scVelo's dynamical model. Lastly the inventors combined topics 3, 4, and 8. Finally, the inventors applied scVelo's dynamical model and visualize the transition matrix on a streamline embedding.

Analysis of the Human Hematopoiesis scNT-Seq Data

This data contains count matrices with or without metabolic labeling information. The inventors focused the analysis on the latter. The inventors used scVelo's stochastic and dynamical models to infer velocities and obtained streamline embeddings. The inventors then applied topic modeling with 8 topics and identified topic genes. The inventors removed topic genes from topics 5 and 6 for downstream analysis because they are strongly associated with minichromosomal and ribosomal genes and are ubiquitously expressed. The inventors then used late integration and use cells above the 35^{th}-percentile for each topic to infer the kinetic parameters and the global transition matrix. The inventors compared the topic-specific velocity to the global velocity from scVelo's dynamical model.

### Analysis of the Mouse Gastrulation Data

After standard preprocessing, the inventors applied scVelo's stochastic model. The inventors did not use the dynamical model because it is more prone to infer transcriptional boost as downregulation [21]. The inventors performed topic modeling with 2 topics and identified 1961 topic genes. The inventors then did more quality control on gene selections by removing genes for which the ratio of the maximum of spliced counts/the maximum of unspliced counts is greater than or less than 0.01. Furthermore, the inventors observed this dataset contains many highly-expressed genes which standard size-normalization steps do not account. To avoid the possibility of not accounting for the dynamics of the lowly expressed genes properly, the inventors used the raw counts to infer the kinetic parameters. The inventors used the early integration strategy to obtain a global transition matrix which was then visualized with standard procedures.

### Analysis of the Human Bone Marrow Data

After standard preprocessing, the inventors applied scVelo's stochastic model. The inventors did not use the dynamical model because it is more prone to characterize transcriptional boost as downregulation [22]. The inventors performed topic modeling with 10 topics. The inventors used late integration and use cells above the 65^{th}-percentile for each topic to infer the kinetic parameters and the global transition matrix.

### Analysis of the Mouse Dentate Gyrus Data

After standard preprocessing, the inventors applied scVelo's dynamical model. The inventors allowed more EM updates than the default parameters for recovering the dynamics and computed the corresponding transition multiple times but were unable to reproduce the oligodendrocyte lineages [5]. The inventors performed topic modeling with 10 topics. The inventors identified topic 1 as a proliferation program characterized by topic genes with high level of expressions across all cells. Thus topic 1 genes were removed for downstream analysis. The inventors used late integration and use cells above the 80^{th}, 90^{th}, 70^{th}, 80^{th}, 60^{th}, 60^{th}, 60^{th}, 90^{th}, and 70^{th}-percentile for topic 0 and topics 2-9 respectively to infer the kinetic parameters and the global transition matrix.

### Analysis of the Mouse Pancreas Data

After standard preprocessing, the inventors applied scVelo's dynamical model. The inventors performed topic modeling with 6 topics. The inventors used late integration and use cells above the 80^{th}, 50^{th}, 60^{th}, 80^{th}, 80^{th}, and 70^{th}-percentile for topics 0 to 5 respectively to infer the kinetic parameters and the global transition matrix.

### Analysis of the Mouse ILC Data

This dataset comes with data from five different days. The inventors only use the data collected on day 3. After standard preprocessing, the inventors performed topic modeling with 10 topics. The inventors applied late integration on topics 4, 6 and 9 with a threshold of 82^{th} percentile for choosing topic cells. The inventors then applied scVelo's stochastic and dynamical model on these topic cells. The inventors focused the velocity comparison on TopicVelo's topic-specific velocity to the global velocity from scVelo's dynamical model.

All of the methods disclosed and claimed herein can be made and executed without undue experimentation in light of the present disclosure. While the compositions and methods of this invention have been described in terms of preferred aspects, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. Kharchenko, P. V. The triumphs and limitations of computational methods for scRNA-seq. Nature Methods 18, 723-732. issn: 1548-7105. http://doi.org/10.1038/s41592-021-01171-x (July 2021).
2. Lähnemann, D. et al. Eleven grand challenges in single-cell data science. Genome Biology 21, 31. issn: 1474-760X. https://doi.org/10.1186/s13059-020-1926-6 (February 2020).
3. Saelens, W., Cannoodt, R., Todorov, H. & Saeys, Y. A comparison of single-cell trajectory inference methods. Nature Biotechnology 37, 547-554. issn: 1546-1696. https://doi.org/10.1038/s41587-019-0071-9 (May 2019).
4. La Manno, G. et al. RNA velocity of single cells. Nature 560, 49498. issn: 1476-4687. https://doi.org/10.1038/01586-018-0414-6 (August 2018).
5. Bergen, V., Lange, M., Peidli, S., Wolf, F. A. & Theis, F. J. Generalizing RNA velocity to transient cell states through dynamical modeling. Nature Biotechnology, 1546-1696. https://doi.org 10.1038/s41587-020-0591-3 (2020).
6. Wolf, F. A. et al. PAGA: graph abstraction reconciles clustering with trajectory inference through a topology preserving map of single cells. Genome Biology 20, 59. issn: 1474-760X. https://doi.org/10.1186/s13059-019-1663-x (March 2019).
7. Street, K. et al. Slingshot: cell lineage and pseudotime inference for single-cell transcriptomics. BMC Genomics 19, 477. issn: 1471-2164. https://doi.org/10.1186/s12864-018-4772-0 (June 2018).
8. Haghverdi, L., Büttner, M., Wolf, F. A., Buettner, F. & Theis, F. J. Diffusion pseudotime robustly reconstructs lineage branching. Nature Methods 13, 845-848. issn: 1548-7105. https://doi.org/10.1038/nmeth.3971 (October 2016).
9. Gorin, G., Svensson, V. & Pachter, L. Protein velocity and acceleration from single-cell multiomics experiments. Genome Biology 21, 39. issn: 1474-760X. https://doi.org/10.1186/s13059-020-1945-3 (February 2020).
10. Li, C., Virgilio, M., Collins, K. L. & Welch, J. D. Single-cell multi-omic velocity infers dynamic and decoupled gene regulation. bioRxiv. eprint: https://www.biorxiv.org.content/early/2021/12/15/2021.12.13.472472.full.pdf. https://www.biorxiv.org.content/early/2021/12/15/2021.12.13.472472 (2021).
11. Lange, M. et al. CellRank for directed single-cell fate mapping. Nature Methods 19, 159 170. https://doi.org/10.1038/s41592-021-01346-6 (2022).
12. Gayoso, A. et al. Deep generative modeling of transcriptional dynamics for RNA velocity analysis in single cells. bioRxiv. eprint: https://www.biorxiv.org.content/early/2022/08/15/2022.08.12.503709.full.pdf. https://www.biorxiv.org.content/early/2022/08/15/2022.08.12.503709(2022).
13. Gorin, G. & Pachter, L. Analysis of Length Biases in Single-Cell RNA Sequencing of Unspliced mRNA by Markov Modeling. Biophysical Journal 120. Publisher: Elsevier, 81a. issn: 0006-3495. https://doi.org/10.1016/j.bpj.2020.11.706 (2021) (February 2021).
14. Qiao, C. & Huang, Y. Representation learning of RNA velocity reveals robust cell transitions. Proceedings of the National Academy of Sciences 118, e2105859118. eprint: https://www.pnas.org/doi/pdf/10.1073/pnas.2105859118. https://www.pnas.org/doi/abs/10.1073/pnas.2105859118 (2021).
15. Gao, M., Qiao, C. & Huang, Y. UniTVelo: temporally unified RNA velocity reinforces single-cell trajectory inference. bioRxiv. eprint: https://www.biorxiv.org/content/early/2022/09/01/2022.04.27.489808.full.pdf. https://www.biorxiv.org/content/early/2022/09/01/2022.04.27.489808 (2022).
16. Farrell, S., Mani, M. & Goyal, S. Inferring single-cell dynamics with structured dynamical representations of RNA velocity. bioRxiv. eprint: https://www.biorxiv.org/content/early/2022/08/23/2022.08.22.504858.full.pdf. https://www.biorxiv.org/content/early/2022/08/23/2022.08.22.504858 (2022).
17. Cui, H., Maan, H., Taylor, M. D. & Wang, B. DeepVelo: Deep Learning extends RNA velocity to multi-lineage systems with cell-specific kinetics. bioRxiv. eprint: https://www.biorxiv.org/content/early/2022/05/30/2022.04.03.486877.full.pdf. https://www.biorxiv.org/content/early/2022/05/30/2022.04.03.486877 (2022).
18. Zhang, Z. & Zhang, X. Inference of high-resolution trajectories in single-cell RNA-seq data by using RNA velocity. Cell Reports Methods 1, 100095. issn: 2667-2375. https://www.sciencedirect.com/science/article/pii/S2667237521001508 (2021).
19. Liu, R., Pisco, A. O., Braun, E., Linnarsson, S. & Zou, J. Dynamical Systems Model of RNA Velocity Improves Inference of Single-cell Trajectory, Pseudo-time and Gene Regulation. Journal of Molecular Biology 434. Artificial Intelligence, Machine Learning and the Changing Landscape of Molecular Biology, 167606. issn: 0022-2836. https://www.sciencedirect.com/science/article/pii/S002228362200186 (2022).
20. Qiu, X. et al. Mapping transcriptomic vector fields of single cells. Cell 185, 690-711.e45. issn: 0092-8674. https://www.sciencedirect.com/science/article/pii/80092867421015774 (2022).
21. Bartle, M. et al. Coordinated changes in gene expression kinetics underlie both mouse and human erythroid maturation. Genome Biology 22. issn: 1474-760X. https://doi.org/10.1186/s13059-021-02414-y (July 2021).
22. Bergen, V., Soldatov, R. A., Kharchenko, P. V. & Theis, F. J. RNA velocity-current challenges and future perspectives. Molecular Systems Biology 17, e10282. issn: 1744 4292. https://doi.org/10.15252/msb.202110282 (August 2021).
23. Pijuan-Sala, B. et al. A single-cell molecular map of mouse gastrulation and early organogenesis. en. Nature 566. issn: 1476-4687. https://doi.org/10.1038/s41586-019-0933-9 (2021) (February 2019).
24. Gorin, G., Fang, M., Chari, T. & Pachter, L. RNA velocity unraveled. PLOS Computational Biology 18, 1-55. https://doi.org/10.1371/journal.pcbi.1010492 (September 2022).
25. Meng, S. C., Stein-O'Brien, G., Boukas, L., Goff, L. A. & Hansen, K. D. Pumping the brakes on RNA velocity—understanding and interpreting RNA velocity estimates. bioRxiv. eprint: https://www.biorxiv.org/content/early/2022/06/25/2022.06.19.494717.full.pdf. https://www.biorxiv.org/content/early/2022/06/25/2022.06.19.494717 (2022).
26. Riba, A. et al. Cell cycle gene regulation dynamics revealed by RNA velocity and deep-learning. Nature Communications 13, 2865. issn: 2041-1723. https://doi.org/10.1038/s41467-022-30545-8 (May 2022).
27. Blei, D. M., Ng, A. Y. & Jordan, M. I. Latent Dirichlet Allocation. Journal of Machine Learning Research 3, 993-1022. (2018) (2003).
28. Blei, D. M. Probabilistic topic models. Science 55, 77-84. https://doi.org/10.1145/2133806.2133826 (2018) (2012).
29. Lee, D. D. & Seung, H. S. Learning the parts of objects by non-negative matrix factorization. Nature 401, 788-791. issn: 1476-4687. https://doi.org/10. 1038/44565 (October 1999).
30. Pritchard, J. K., Stephens, M. & Donnelly, P. Inference of Population Structure Using Multilocus Genotype Data. Genetics 155, 945-959. issn: 1943-2631. https://doi.org/10.1093/genetics/155.2.945 (2021) (June 2000).
31. Erosheva, E. A. in Bayesian Statistics 7 (eds Bernardo, J. M. et al.) 501-510 (Oxford University Press, Oxford, 2003).
32. Bielecki, P. et al. Skin-resident innate lymphoid cells converge on a pathogenic effector state. Nature 592, 128-132. issn: 1476-4687. https://doi.org/10.1038/s41586-021-03188-w (April 2021).
33. Dey, K. K., Hsiao, C. J. & Stephens, M. Visualizing the structure of RNA-seq expression data using grade of membership models. PLoS genetics 13, e1006599. issn: 1553-7404. https://doi.org/10.1371/journal.pgen.100599 (March 2017).
34. Dani, N. et al. A cellular and spatial map of the choroid plexus across brain ventricles and ages. Cell 184, 3056-3074.e21. issn: 0092-8674. https://www.sciencedirect.com/science/article/pii/S0092867421004384 (2021).
35. Mao, Y., Cai, H., Zhang, Z., Tang, J. & Li, Y. Learning interpretable cellular and gene signature embeddings from single-cell transcriptomic data. Nature Communications 12, 5261. issn: 2041-1723. https://doi.org/10.1038/s41467-021-25534-2 (September 2021).
36. Singh, A. & Bokes, P. Consequences of mRNA Transport on Stochastic Variability in Protein Levels. Biophysical Journal 103, 1087-1096. issn: 0006-3495. https://www.sciencedirect.com/science/article/pii/S0006349512007904 (2012).
37. Gorin, G. & Pachter, L. Length Biases in Single-Cell RNA Sequencing of pre-mRNA. bioRxiv, 2021.07.30.454514. https://doi.org/10.1101/2021.07.30.454514 (July 2021).
38. Wilk, A. J. et al. A single-cell atlas of the peripheral immune response in patients with severe COVID-19. Nature Medicine 26, 1070-1076. issn: 1546-170X. https://doi.org/10.1038/s41591-020-0944-y (July 2020).
39. Alquicira-Hernandez, J., Powell, J. E. & Phan, T. G. No evidence that plasmablasts transdifferentiate into developing neutrophils in severe COVID-19 disease. eng. Clinical & translational immunology 10. CTI21308[PII], e1308-e1308. issn: 2050-0068. https://doi.org/10.1002/cti2.1308 (June 2021).
40. Wilk, A. J. et al. Additional analyses exploring the hypothesized transdifferentiation of plasmablasts to developing neutrophils in severe COVID-19. bioRxiv. eprint: https://www.biorxiv.org/content/early/2020/10/16/2020.10.15.339473.full.pdf. https://www.biorxiv.org/content/early/2020/10/16/2020.10.15.339473 (2020).
41. Carbonetto, P., Sarkar, A., Wang, Z. & Stephens, M. Non-negative matrix factorization algorithms greatly improve topic model fits. arXiv 2105.13440. arXiv: 2105.13440. https://arxiv.org/abs/2105.13440 (2021).
42. Xu, S., Zhao, L., Larsson, A. & Venge, P. The identification of a phospholipase B precursor in human neutrophils. en. FEBS J 276, 175-186 (November 2008).
43. Levens, D. & Larson, D. R. A new twist on transcriptional bursting. eng. Cell 158. S0092-8674(14)00869-1 [PII], 241-242. issn: 1097-4172. https://doi.org/10.1016/j.cell.2014.06.042 (July 2014).
44. Cao, J., Xia, T., Li, J., Zhang, Y. & Tang, S. A density-based method for adaptive LDA model selection. Neurocomputing 72. Advances in Machine Learning and Computational Intelligence, 1775-1781. issn: 0925-2312. https://www.sciencedirect.com/science/article/pii/S092523120800372X (2009).
45. Deveaud, R., SanJuan, E. & Bellot, P. Accurate and effective latent concept modeling for ad hoc information retrieval. Document numérique 17, 61-84 (April 2014).
46. Röder, M., Both, A. & Hinneburg, A. Exploring the Space of Topic Coherence Measures in Proceedings of the Eighth ACM International Conference on Web Search and Data Mining (Association for Computing Machinery, Shanghai, China, 2015), 399-408. isbn: 9781450333177. https://doi.org/10.1145/2684822.2685324.
47. Songdej, N. et al. Transcription Factor RUNX1 Regulates Factor FXIIIA Subunit (F13A1) Expression in Megakaryocytic Cells and Platelet F13A1 Expression is Downregulated in RUNX1 Haplodeficiency. Blood 136, 25-26. issn: 0006-4971. https://doi.org/10.1182/blood-2020-141382 (November 2020).
48. Psaila, B. et al. Single-Cell Analyses Reveal Megakaryocyte-Biased Hematopoiesis in Myelofibrosis and Identify Mutant Clone-Specific Targets. Molecular Cell 78, 477 492.e8. issn: 1097-2765. https://www.sciencedirect.com/science/article/pii/S1097276520302343 (2020).
49. Shim, M.-H., Hoover, A., Blake, N., Drachman, J. G. & Reems, J. A. Gene expression profile of primary human CD34+CD38lo cells differentiating along the megakaryocyte lineage. Experimental Hematology 32, 638-648. https://doi.org/10.1016/j.exphem.2004.04.002 (July 2004).
50. Li, Y., Qi, X., Liu, B. & Huang, H. The STAT5-GATA2 pathway is critical in basophil and mast cell differentiation and maintenance. en. J Immunol 194, 4328-4338 (March 2015).
51. Karlsson, M. et al. A single-cell type transcriptomics map of human tissues. Science Advances 7. https://doi.org/10.1126/sciadv.abh2169 (July 2021).
52. Setty, M. et al. Characterization of cell fate probabilities in single-cell data with Palantir. Nature Biotechnology 37. Publisher. Nature Publishing Group, 451-460. issn: 15461696. https://doi.org/10.1038/s41587-019-0068-4 (April 2019).
53. Suzuki, M. et al. GATA factor switching from GATA2 to GATA1 contributes to erythroid differentiation. Genes to Cells 18, 921-933. eprint: https://onlinelibrary.wiley.com/doi/pdf/10.1111/gtc.12086. https://onlinelibrary.wiley.com/doi/abs/10.1111/gtc.12086 (2013).
54. Tusi, B. K. et al. Population snapshots predict early haematopoietic and erythroid hierarchies. en. Nature 555, 54-60 (February 2018).
55. Yan, H. et al. Developmental differences between neonatal and adult human erythropoiesis. American Journal of Hematology 93, 494-503. eprint: https://onlinelibrary.wiley.com/doi/pdf/10.1002/ajh.25015. https://onlinelibrary.wiley.com/doi/abs/10.1002/ajh.25015 (2018).
56. Sichien, D. et al. IRF8 Transcription Factor Controls Survival and Function of Terminally Differentiated Conventional and Plasmacytoid Dendritic Cells, Respectively. en. Immunity 45, 626-640 (September 2016).
57. Wang, H. et al. Decoding Human Megakaryocyte Development. Cell Stem Cell 28, 535-549.e8. issn: 1934-5909. https://www.sciencedirect.com/science/article/pii/S1934590920305440 (2021).
58. Pellin, D. et al. A comprehensive single cell transcriptional landscape of human hematopoietic progenitors. Nature Communications 10, 2395. issn: 2041-1723. https://doi.org/10.1038/s41467-019-10291-0 (June 2019).
59. Chertov, O. et al. Identification of human neutrophil-derived cathepsin G and azurocidin/CAP37 as chemoattractants for mononuclear cells and neutrophils. en. J Exp Med 186, 739-747 (August 1997).
60. Hochgerner, H., Zeisel, A., Lönnerberg, P. & Linnarsson, S. Conserved properties of dentate gyrus neurogenesis across postnatal development revealed by single-cell RNA sequencing. Nature Neuroscience 21, 290-299. issn: 1546-1726. https://doi.org/10.1038/s41593-017-0056-2 (February 2018).
61. Bastidas-Ponce, A. et al. Comprehensive single cell mRNA profiling reveals a detailed roadmap for pancreatic endocrinogenesis. Development 146. dev173849. issn: 0950 1991. eprint: https://journals.biologists.com/dev/article-pdf/146/12/dev173849/1857007/dev173849.pdf. https://doi.org/10.1242/dev.173849 (June 2019).
62. Cahoy, J. D. et al. A transcriptome database for astrocytes, neurons, and oligodendrocytes: a new resource for understanding brain development and function. en. J Neurosci 28, 264-278 (January 2008).
63. Yuelling, L. W., Waggener, C. T., Afshari, F. S., Lister, J. A. & Fuss, B. Autotaxin/ENPP2 regulates oligodendrocyte differentiation in vivo in the developing zebrafish hindbrain. Glia 60, 1605-1618. https://doi.org/10.1002/glia.22381 (July 2012).
64. Muraro, M. J. et al. A Single-Cell Transcriptome Atlas of the Human Pancreas. en. Cell Syst 3, 385-394.e3 (September 2016).
65. Xin, Y. et al. RNA Sequencing of Single Human Islet Cells Reveals Type 2 Diabetes Genes. Cell Metabolism 24, 608-615. issn: 1550-4131. https://www.sciencedirect.com/science/article/pii/S155041311630434X (2016).
66. Byrnes, L. E. et al. Lineage dynamics of murine pancreatic development at single-cell resolution. Nature Communications 9, 3922. issn: 2041-1723. https://doi.org/10.1038/s41467-018-06176.3 (September 2018).
67. Vivier, E. et al. Innate Lymphoid Cells: 10 Years On. en. Cell 174, 1054-1066 (August 2018).
68. Cao, Z., Sun, X., Icli, B., Wara, A. K. & Feinberg, M. W. Role of Kruppel-like factors in leukocyte development, function, and disease. en. Blood 116, 4404414 (July 2010).
69. Lambert, S. A. et al. The Human Transcription Factors. Cell 172, 650-665. issn: 0092 8674. https://www.sciencedirect.com/science/article/pii/S0092867418301065 (2018).
70. Pokhilko, A. et al. Targeted single-cell RNA sequencing of transcription factors enhances the identification of cell types and trajectories. en. Genome Res. 31, 1069-1081 (June 2021).
71. Gorin, G., Vastola, J. J., Fang, M. & Pachter, L. Interpretable and tractable models of transcriptional noise for the rational design of single-molecule quantification experiments. bioRxiv. eprint: https://www.biorxiv. org/content/early/2021/12/26/2021.09.06.459173.full.pdf. https://www.biorxiv.org/content/early/2021/12/26/2021.09.06.459173 (2021).
72. Soneson, C., Srivastava, A., Patro, R. & Stadler, M. B. Preprocessing choices affect RNA velocity results for droplet scRNA-seq data. PLOS Computational Biology 17, 1-26. https://doi.org/10.1371/journal.pchi.1008585 (January 2021).
73. Stephens, M. False discovery rates: a new deal. Biostatistics 18, 275-294. issn: 1465 4644. eprint: https://academic.oup.com/biostatistics/article-pdf/18/2/275/11057424/kxw041.pdf. https://doi.org/10.1093/biostatistics/kxw041 (October 2016).
74. Lee, M. bab2 min/tomotopy: 0.12.3 version v0.12.3. July 2022. https://doi.org/10.5281/zenodo.6868418.
75. Li, T., Shi, J., Wu, Y. & Thou, P. On the Mathematics of RNA Velocity I: Theoretical Analysis. CSIAM Transactions on Applied Mathematics 2, 1-55. issn: 2708-0579. http://global-sci.org/intro/article_detail/csiam-am/18653.html (2021).
76. Gillespie, D. T. A general method for numerically simulating the stochastic time evolution of coupled chemical reactions. Journal of Computational Physics 22, 403-434. issn: 0021-9991. https://www.sciencedirect.com/science/article/pii/0021999176900413 (1976).
77. Lam, S. K., Pitrou, A. & Seibert, S. Numba: A llvm-based python jit compiler in Proceedings of the Second Workshop on the LLVM Compiler Infrastructure in HPC (2015), 1-6.
78. Virtanen, P. et al. SciPy 1.0: Fundamental Algorithms for Scientific Computing in Python. Nature Methods 17, 261-272 (2020).

1. A method of building a model of cellular trajectory in a plurality of cells, the method comprising:
receiving single cell RNA-sequencing (scRNA-seq) data from each cell in the plurality of cells;
computing the model for the plurality of cells based on the scRNA-seq data using topic modeling and calculating at least one RNA velocity parameter.

2. The method of claim 1, wherein the scRNA-seq data is filtered by removing genes that do not have spliced and unspliced transcripts in at least 20 cells of the plurality of cells.

3. The method of claim 1, wherein the topic modeling comprises applying a topic model to the scRNA-seq data.

4. The method of claim 3, wherein the topic model comprises a multinomial topic model.

5. The method of claim 1, wherein the topic modeling comprises drawing xi for each cell in the plurality of cells, wherein (equation 2) xi1, . . . , xiM|ti˜Multinom(ti; πi1, . . . , πiM)∀1≤i≤C, where C is the number of cells, M is the number of genes, xᵢₘ is number of mRNA transcripts for the m^{th} gene in the i^{th} cell and tᵢ=Σₘ₌₁^{M}x_{iM}.

6. The method of claim 5, wherein (equation 3) π_{iM}=Σₖ₌₁^{K}LᵢₖFₘₖ.

7. The method of claim 1, wherein an optimal number of topics is calculated for the topic modeling.

8. (canceled)

9. The method of claim 1, wherein calculating at least one RNA velocity parameter comprises an RNA velocity parameter estimation by a one-state model.

10. The method of claim 1, wherein calculating at least one RNA velocity parameter comprises an RNA velocity parameter estimation by a geometric burst model.

11. A method of building a model to distinguish cell types in a plurality of cells, the method comprising
receiving single cell RNA-sequencing (scRNA-seq) data from each cell in the plurality of cells;
fitting a topic model to a counts matrix from the scRNA-seq data to generate at least one topic, wherein the counts matrix comprises spliced and unspliced transcript data; and
fitting a geometric burst model to the scRNA-seq data, wherein the burst model finds the probability of observing a cell with unspliced transcripts and spliced transcripts at a specific time.

12. The method of claim 11, wherein the topic model comprises a set number of topics.

13. The method of claim 11, wherein the set number of topics is a calculated optimal number of topics.

14. (canceled)

15. The method of claim 11, further comprising integrating process-specific transition matrices by characterizing a probabilistic flow of topic-specific transcriptional changes across a population of topic-associated cells.

16. The method of claim 11, wherein the spliced and unspliced transcript data comprises data from topic-specific genes.

17. The method of claim 16, wherein the topic-specific gene data is combined across the topic(s) and a global transition matrix is computed.

18. The method of claim 16, wherein the topic-specific genes are genes having a log fold change greater than 0.5 or less than −0.5 and a linear-feedback shift register less than 0.001 in the scRNA-seq data.

19. The method of claim 11, wherein the burst model uses the rate of a Poisson process governing a burst event, the probability of producing the unspliced transcript during a single burst event governed by a geometric distribution, a splicing rate of the unspliced transcripts, and a degradation rate of the spliced transcripts.

20. The method of claim 11, wherein at least one parameter of the geometric burst model is optimized by a Gillespie algorithm.

21. (canceled)

22. The method of claim 11, wherein at least one parameter of the geometric burst model is optimized by a Nelder-Mead algorithm.

23.-28. (canceled)

29. A system for building a model of cellular trajectory in a plurality of cells, comprising:
a database storing single cell RNA-sequencing (scRNA-seq) data; and
one or more computer processors operatively couple to said database wherein said one or more computer processors are individually or collectively programmed to perform the steps of claim 1.

30.-32. (canceled)
